(19) United States
(12) Patent Application Publication
(10) Pub. No.: US 2010/0112020 A1
(43) Pub. Date: May 6, 2010

(54) NANOFIBROUS STRUCTURES AND THEIR USE IN DENTAL APPLICATIONS

(76) Inventors: Philippe Westbroek, Dendermonde (BE); Tamara Van Camp, Gent (BE); Sander De Vrieze, Eine (BE); Karen De Clerck, Sint-Amandsberg (BE); Jan Braem, Sint-Kruis-Brugge (BE); Jean De Dieu Hakuzimana, Wortegem-Petegern (BE); Emmanuel Gasana, Gent (BE)

Correspondence Address: BACON & THOMAS, PLLC, 625 SLATERS LANE, FOURTH FLOOR, ALEXANDRIA, VA, 22314-1176, US

(21) Appl. No.: 12/530,417

(22) PCT Filed: Mar. 10, 2008
(86) PCT No.: PCT/EP08/52835

§ 371 Date: Sep. 8, 2009

(30) Foreign Application Priority Data
Mar. 9, 2007 (GB) 0704615.4
May 18, 2007 (EP) 07009995.7
Dec. 7, 2007 (EP) 07076056.6

(51) Int. Cl. A61K 8/02; B29C 47/30; B27N 5/00; B32B 5/24; A61Q 11/00; B01D 61/00

(52) U.S. Cl. 424/401; 425/174.8 E; 264/465; 424/49; 210/767

(57) ABSTRACT

An electrospinning device is described for producing nanofibrous porous structures. The device comprises three or more neighbouring outlets (11) separated from one another by a distance of at least 4 cm and positioned in a first plane (12). The device also comprises a second surface (13) for receiving output from the outlets. A relative movement between the three or more neighbouring outlets (11) and the second surface (13) can be applied in a first direction and second direction, the second direction being substantially perpendicular to the first direction. The second surface (13) thereby is facing the first plane wherein the outlets are positioned. The device furthermore comprises a voltage source (9) adapted to apply a potential difference between the first surface and the second surface. It furthermore may comprise a recipient for containing a solution or melt to be electrospun from the outlets and comprises a providing means for providing (10) the solution or melt to the outlets. The present invention in one aspect also relates to a teeth whitening system for whitening teeth. The system is based on a nano-fibrous structure for applying to the teeth and also comprises a teeth whitening moiety comprising a bleaching agent. The use of the nano-fibrous structure allows the use of a low viscosity teeth whitening moiety, a good fit of the structure over the teeth, thus allowing to treat the tips of the canine teeth and sticking the device to the teeth whereby the preferred shape of the device when in use can be maintained. The present invention also relates to a corresponding nano-fibrous structure and a method for bleaching teeth.

### TECHNICAL FIELD OF THE INVENTION

The present invention relates to a fibrous structure, a process and a device for manufacturing the same. In particular, the present invention relates to fibrous structures such as e.g. nanofibrous structures and their applications as absorption or filtration material such as in wound dressings, wipers, diapers, filtration, compound immobilization, inclusion of chemicals, etc. In one particular aspect, the present invention relates to the field of dental applications. More particularly, the present application relates to methods, products and systems for whitening of teeth applications.

### BACKGROUND OF THE INVENTION

Nanofibrous structures are useful in a variety of applications in the fields of clothing, filtering, medicine, cosmetics and defense. There is a strong interest in nanofibrous structures based on their high porosity for absorption, immobilization and inclusion of chemicals, solvent, solutions, melts and liquid phases. In many applications, such as wound burn treatment, teeth whitening gel immobilization, disinfecting solution immobilization, urine absorption and other applications where high absorption is preferred, an absorption capacity of about 5 mL·cm⁻² is preferably obtained. In the same applications large nanofibrous structures (e.g. for wound burn treatment a minimum dimension is 40 by 40 cm for a mat) are needed. In order to guarantee homogeneous absorption behaviour over the structure it is useful to obtain a regular thickness over the entire structure.

Nanofibrous structures can be produced using an electrospinning setup. A basic setup is shown in FIG. 1 and consists of a high voltage source 1, an anesthesia pump 2, the pump comprising a syringe 3 that contains a polymer solution 4 and the pump transporting polymer solution towards the tip of a metallic needle 5, said needle positioned in a spinneret 6, said spinneret comprising an upper 7 and a lower 8 conductive plate. An electrical field is applied over the upper and lower plate resulting in an extrusion ability of the polymer solution at the tip of the needle towards the surface of the lower element. The electrical field created, causes the polymer solution to overcome the cohesive forces that hold the polymer solution together. As a result of cohesive force compensation by the electrical field a jet will be drawn from the polymer solution droplet, which forms nano-dimensioned fibres, finally collected at the lower plate. Typical dimensions of the deposited structures are circular surfaces with a diameter of about 10 to 15 cm. Therefore, with a single nozzle system, it is not possible to obtain the large surface areas required for many applications in an economic feasible way.

Multi-nozzles apparatus have been described as attempts to enable upscaling of the electrospining process. Such a device is described in International patent application WO 2005/073442. In WO 2005/073442, a multi-nozzle electrospinning device is disclosed wherein a continuous nanofiber filament is formed by producing and twisting a nanofiber web. WO 2005/073442 does not disclose means to achieve the production of nanofibrous structures composed of straight fibers and/or non-crosslinked fibers and/or fibers of controlled diameter. Furthermore WO 2005/073442 does not disclose means to achieve the production of fibrous structures having a uniform thickness and/or a large width and/or a high strength.

There is therefore still a need in the art for a device and a method to produce fibrous (e.g. nanofibrous) structures with high porosity, large width, high mechanical strength and uniform thickness in an economic viable way. There is also a need for a device and a method to produce fibrous (e.g. nanofibrous) structures with enhanced filtration or liquid transportation properties.

As indicated above, nanofibrous structures can be used in a variety of applications, such as for example in dental applications like teeth bleaching. For bleaching of teeth, two major categories of aesthetic applications are known being applications performed at the dental practice and applications which can be performed outside the dental practice, for example at the consumer's home or at any suitable place. Some of these solutions require a plurality of visits to the dental practice.

Some solutions involving applications which can be performed outside the dental practice, relate to the use of tray made to fit the mouth and teeth of the user, which is made at the dental practice but which can be used at home. Such a device typically may need to be re-used in view of the cost and must be robust in order to allow repeatedly handling, cleaning, filling, installation, removal, etc.

Low cost solutions also have been provided, wherein a one-size-fits-all system is used. As these systems often do not result in a perfect fit to the teeth, the amount of bleaching agent provided often is increased. On the other hand, such systems also suffer from leakage of the bleaching agent to the Gingiva and optionally to ingestion.

US2005/0196352 A1 by the Proctor and Gamble Company discloses a teeth bleaching method whereby the method includes applying a tooth bleaching delivery system to a plurality of adjacent teeth. The tooth bleaching delivery system includes a strip of material and a tooth bleaching composition having a peroxide active component. The method includes applying a first portion of the strip material to the facial surfaces of the teeth, folding a second portion of the strip of the material about the incisal edges of the plurality of adjacent teeth and folding a second portion of the strip material over incisal edges of the adjacent teeth to apply the second portion to the lingual surfaces of the teeth.

### SUMMARY OF THE INVENTION

It is an object of the present invention to provide good devices or methods for producing fibrous (e.g. nanofibrous) structures. It is an advantage of embodiments according to the present invention that fibrous structures with high porosity are provided and methods for producing them. It is an advantage of embodiments according to the present invention that fibrous structures with large width and/or uniform thickness are provided and methods for producing them. It is also an advantage of embodiments according to the present invention that fibrous structures with improved mechanical strength are provided and methods for producing them. It is furthermore an advantage of embodiments according to the present invention that fibrous structures with good liquid uptake are provided and methods for producing them. It is also an advantage of embodiments according to the present invention that fibrous structures with good control release and filtration properties are provided and methods for producing them. It is an advantage of embodiments according to the present invention that fibrous structures can be provided in an economic viable way. It is an advantage of embodiments according to the present invention that fibrous structures with a combination of two or more of the above described advantages can be obtained.

The above objective is accomplished by a method and device according to the present invention.

The invention relates to an electrospinning device for producing fibrous structures, said electrospinning device comprising a set of three or more outlets, advantageously neighbouring outlets, for outputting solution or melt, said three or more outlets being arranged in a first plane, a second planar surface arranged parallel to said first plane, the second planar surface for receiving output from said three or more outlets, wherein said set of three or more outlets and said second planar surface are adapted to move relatively to each other, a voltage source for generating a potential difference between said set of three or more outlets and said second surface, providing means for providing said solution or melt to said outlets, characterized in that each of said three or more outlets are separated from one another by a distance of at least 4 cm. It is an advantage of embodiments according to the present invention that devices are provided allowing to produce fibrous structures with high porosity. At least two neighbouring outlets of said three or more outlets may be separated by a distance of at least 6 cm. It is particularly advantageously to separate two neighbouring outlets by a distance of at least 4 cm. Each two neighbouring outlets of said at least three or more outlets may be separated from one another by a distance of at least 6 cm, advantageously a distance of at least 8 cm. In other words, the distance to the closest other nozzle may be at least 4 cm, advantageously at least 6 cm. Each two of said at least three or more outlets may be separated from one another by a distance of at least 6 cm. Other nozzles not neighbouring the set of three or more neighbouring nozzles, i.e. at further distance to the set of neighbouring nozzles may also be present, and between these other nozzles, other internozzle distances may occur. It is an advantage of embodiments according to the present invention that devices are provided allowing production of fibrous structures that are strong, have high porosity and straight fibres. It is an advantage of embodiments according to the present invention that devices are provided allowing production of fibrous structures that are strong, have high porosity and straight fibres. As an optional feature, the outlets (e.g. needles) are positioned in a triangle setup or a multiple thereof.

The set of three or more neighbouring outlets may be arranged in different rows, outlets of adjacent rows may be in a staggered configuration.

The set of three or more neighbouring outlets may be arranged in different rows being parallel but shifted with respect to their corresponding position of the outlets in the rows.

The device according to any of the previous claims, wherein outputs of said three or more neighbouring outlets are arranged in different rows, wherein the distance between a first row and a second row, adjacent to said first row, is at least 1.5 times larger than the distance between a first row and a third row, the third row being adjacent to said first row but positioned at the opposite side of the first row compared to said second row.

The distance between each of said three or more outlets may be adapted for obtaining a fibrous structure comprising at least 50% of fibers substantially free of cross-links to neighboring fibers. It is an advantage of embodiments according to the present invention that devices are provided allowing production of fibrous structures wherein only a low degree of cross-linked fibers is present. Cross-link free thereby may be that there is absence of covalent bonds linking one polymer chain of one fibre to another polymer chain of a neighbouring fibre. The distance between the three or more outlets also may be adapted for obtaining a fibrous structure comprising at least 50% of fibres substantially free of any chemical bound. The distance between each of said three or more outlets may be adapted for obtaining a fibrous structure comprising at least 50% of straight fibers. The device may be adapted for applying a relative movement between said three or more outlets and said second surface in a first direction and in a second direction, different from said first direction. The second direction may be perpendicular to said first direction. The second direction may be parallel to said first plane. Alternatively, the second direction may be perpendicular to said first plane. The three or more outlets may be adapted to be movable reciprocally in said first direction. In embodiments of the present invention, the device may be adapted for applying one or more relative movements between said set of outlets and said receiving surface, e.g. at any stage of the processing. In embodiments of the present invention, the set of outlets may be adapted to be movable reciprocally in a direction parallel to said receiving surface and perpendicular to said first direction. This is advantageous because it permits the outputted umbrellas of solution or melts to overlap on the receiving surface.

The device may comprise control means for varying the diameter of the produced fibres.

The means for varying the diameter of the produced fibres may be control means for altering the distance between said first plane and said second planar surface during the production of the fibrous structure.

The device may be adapted for generating a plurality of fibers, whereby at least 50% of said plurality of fibers may comprise an average diameter between 3 and 2000 nm. The device may be adapted for using a polymer solution or melt comprising at least one of a polyimide, polystyrene, polycaprolactone, polyacrylonitrile, polyethylene oxide, polylactic acid, polyacrylic acid, polyesteramide, polyvinyl alcohol, polyimide, polyurethane, polyvinylpyrrolidon, collagen, cellulose, chitosan, methacrylates, silk, polyethylene vinylacetate co polymer, polyethylene vinylalcohol copolymer, polyvinylbutyral or metal.

In embodiments of the present invention, the device may further comprise a recipient for containing a solution or melt to be electrospun from said outlets, and means for providing said solution or melt to said outlets. As an optional feature, said recipient may contain a polymer solution or melt. As an optional feature, the device may allow the production of nanofibrous structures with a width between 15 cm and 10.000 cm. As another optional feature, the device may comprise a surrounding element over the spinneret to avoid instability and to allow solvent removal and recuperation. As another optional feature, the device may comprise a temperature control system that allows to control the temperature in the range of 280-1500 K.

The present invention also relates to a method for producing fibrous structures, said method comprising the steps of moving a set of three or more neighbouring outlets, for outputting solution or melt, said set being comprised in a first plane, relatively to a second planar surface, for receiving output of said three or more neighbouring outlets, applying a potential difference between said set of three or more neighbouring outlets and said second planar surface, and, during said moving and applying, providing a solution or melt to said outlets, wherein each two neighbouring outlets of said set of three or more neighbouring outlets are separated from one another by a distance of at least 4 cm. Each two of said at least three or more outlets may be separated from one another by a distance of at least 6 cm. Each two of said at least three or more outlets may be separated from one another by a distance of at least 8 cm. Other nozzles not neighbouring the set of three or more neighbouring nozzles, i.e. at further distance to the set of neighbouring nozzles may also be present, and between these other nozzles, other internozzle distances may occur.

As an advantageous optional feature, the method may further comprise the step of moving reciprocally at least one of said set of outlets and/or said receiving surface in a direction parallel to said receiving surface and perpendicular to said first direction. The moving step may comprise adapting the distance between said set of three or more outlets and said second planar surface. The variation of the distance between the outlets and the receiving surface may optionally be obtained by adapting the distance between said neighbouring outlets and said receiving surface during the production of the fibrous structure.

The distance may be adapted by providing a relative movement between said set of outlets and said second planar surface in a first direction and a second direction perpendicular to said first direction and perpendicular to said first plane. The moving step may be performed such as to achieve a predefined distance between said set of outlets and said second planar surface, and, while performing the applying step and providing step, at least one of said set of outlets and/or said second planar surface may be moved reciprocally in a first direction and in a second direction perpendicular to the first direction and parallel to said first plane. The method furthermore may comprise iterating the first moving step and the reciprocally moving step for a predetermined number of times, wherein the sense of movement is reversed between each iteration.

The method may be adapted for generating a plurality of fibers, whereby at least 50% of said plurality of fibers comprises an average diameter between 3 and 2000 nm. The method may be adapted for using a polymer solution or melt comprising at least one of a polyamide, polystyrene, polycaprolactone, polyacrylonitrile, polyethylene oxide, polylactic acid, polyacrylic acid, polyesteramide, polyvinyl alcohol, polyimide, polyurethane, polyvinylpyrrolidon, collagen, cellulose, chitosan, methacrylates, silk, polyethylene vinylacetate co-polymer, polyehthylene vinylalcohol copolyper, polyvinylbutyral or metal.

As an optional feature, a voltage difference of between 100 V and 200000 V may be applied over the set of outlets and the receiving surface.

As another optional feature, the pump rate of the polymer solution or melt per outlet may be between 0.01 and 500 mL h⁻¹.

As an optional feature, the solutions or melts may contain an additional compound, such as compounds with antibacterial, pharmaceutical, hydrophobic/hydrophilic, anti corrosion, catalytic, oxidative/reductive and other properties.

The present invention also relates to an electrospun fibrous structure manufactured using a method according to embodiments of the present invention as described above.

The present invention relates to an electrospun fibrous structure, the structure comprising at least 50% of fibres having segments substantially straight over a distance of 5 μm, in the present application referred to as straight fibres.

The electrospun fibrous structure may comprise at least 50% of fibers that is substantially cross-link free with respect to neighboring fibers. Cross-link free thereby may be that there is absence of covalent bonds linking one polymer chain of one fibre to another polymer chain of a neighbouring fibre. The distance between the three or more outlets also may be adapted for obtaining a fibrous structure comprising at least 50% of fibres substantially free of any chemical bound.

The electrospun fibrous structure may at least 50% of randomly oriented fibers.

The electrospun fibrous structure may have a porosity of at least 65%.

50% or more of its fibers may have an average diameter between 3 and 2000 nm, advantageously equal or above 10 nm and/or advantageously equal or below 700 nm.

The present invention also relates to an electrospun fibrous structure comprising two or more layers, wherein each of said layers is composed of fibers having an average diameter different from the average diameter of the fibers of an adjacent layer.

The present invention also relates to the use of an electrospun fibrous structure as described above for filtration or absorption applications.

The present invention also relates to the use of an electrospun fibrous structure as described above for teeth bleaching.

The present invention furthermore relates to a filtrating or an absorbing device comprising an electrospun fibrous structure as described above.

The present invention also relates to a controller for controlling an electrospinning device, the controller being adapted for performing any of the above described methods.

In one aspect, the present invention relates to an electrospinning device for producing nanofibrous structures having a porosity of at least 65%, said electrospinning device comprising a first surface comprising three or more outlets, said first surface being adapted to be movable in a first direction, a second surface adapted to be movable in a second direction, e.g. at an angle to the first direction such as optionally substantially perpendicular to said first direction, said second surface facing said first surface, a voltage source adapted to apply a potential difference between said first surface and said second surface, a recipient for containing a solution or melt to be electrospun from said outlets, and means for providing said solution or melt to said outlets. As an optional feature, the three or more outlets can be separated from one another by a distance of at least 4 cm.

As an optional feature, said recipient may contain a polymer solution or melt.

As an optional feature of this first embodiment, the spinneret may allow the production of nanofibrous structures with a width between 15 and 10.000 cm.

As an optional feature, the non-woven nanofibrous structures comprise nanofibers having a diameter between 10 and 2000 nm, advantageously below 700 nm.

As another optional feature, the lower and the upper section are adapted to be moveable perpendicularly to each other.

As another optional feature, a voltage difference of between 100 V and 200000 V is applied over the upper and lower element of the spinneret.

As another optional feature, the spinneret further comprises a set of needles positioned at the upper plate, said needles releasing a polymer solution or a polymer melt flow, obtained through a pump.

As another optional feature, the needles are positioned in a triangle setup or a multiple thereof.

As another optional feature, the distance between the needles is between 4 and 100 cm.

As another optional feature, the pump rate of the polymer solution or melt per needle is between 0.01 and 500 mL h⁻¹.

As another optional feature, the polymer solution or melt used contains one of the following polymers or polymer classes: polyamides, polystyrenes, polycaprolactones, polyacrylonitriles, polyethylene oxides, polylactic acids, polyacrylic acids, polyesteramides, polyvinyl alcohols, polyimides, polyurethanes, polyvinylpyrrolidon, collagen, cellulose and related products, chitosan, methacrylates, silk, polyethylene vinylacetate copolymer, polyethylene vinylalcohol copolymer, polyvinylbutyral and metal containing nanofibers.

As an optional feature, the solutions or melts may contain an additional compound, such as compounds with antibacterial, farmaceutical, hydrophobic/hydrophilic, anti corrosion, catalytic, oxidative/reductive and other properties.

As another optional feature, structures of nanofibers are obtained with a porosity between 65 and 99%

As another optional feature, structures of nanofibers are obtained, said fibers having a diameter between 3 and 800 nm.

As another optional feature, the device comprises a surrounding element over the spinneret to avoid instability and to allow solvent recuperation.

As another optional feature, the device comprises a temperature control system that allows to control the temperature in the range of 280-1500 K.

In a further aspect, the present invention relates to a method for producing nanofibrous structures having a porosity of at least 65%, said method comprising the steps of applying a potential difference between a first surface and a second surface, said first surface comprising three or more outlets, moving said first surface in a first direction while simultaneously moving said second surface in a second direction substantially perpendicular to said first direction, and providing a solution or melt to said outlets.

The three or more outlets are advantageously separated from one another by a distance of at least 4 cm.

In a third aspect, the present invention relates to nanofibrous polymeric structures having a porosity of at least 65% and a width comprised between 15 and 10000 cm.

In some embodiments, the present invention relates to a device to produce nanofibrous structures (see FIG. 2, FIG. 3 and FIG. 4) from polymer solutions and polymer melts. The device comprises a high voltage source, a spinneret, said spinneret comprising a number of outlets such as e.g. needles, an upper, and a lower element (e.g. surface), means such as but not limited to a peristaltic pump or anesthesia pump for providing the solution or melt to the outlets, an optional surrounding element, and an optional temperature control system, said system may for instance comprise jacketed tubes and containers for liquid or oil based temperature control and gas heaters for environmental temperature control.

In another aspect, the invention relates to nanofibrous structures for dental applications. It is also an object of the present invention to provide good methods and systems for whitening of teeth, also referred to as teeth bleaching. It is an advantage of embodiments according to the present invention that methods and systems allow accurate delivery of teeth bleaching moiety or components thereof. It is an advantage of embodiments according to the present invention that methods and systems are provided allowing little or no leakage to the area of the mouth surrounding the teeth. It is an advantage of embodiments according to the present invention that delivery of teeth bleaching moiety or components thereof can be provided to the canine teeth. It is an advantage of embodiments according to the present invention that a system is provided allowing stable positioning and/or fixation of the device on the teeth of a user. It is an advantage of embodiments according to the present invention that methods and systems are provided requiring less components or particular properties of the bleaching moiety for fixing the device to the teeth, e.g. avoiding the need for a fixating component in the teeth bleaching moiety. It is an advantage of embodiments according to the present invention that methods and systems are provided allowing the use of a low viscosity teeth bleaching, allowing more efficient whitening of teeth.

It is an advantage of embodiments according to the present invention that devices can be made providing a good fit while allowing to also cover the canine teeth. It is an advantage of embodiments according to the present invention that the teeth can be whitened in a relative smooth and/or uniform way. It is an advantage of embodiments according to the present invention that a row of front teeth, including the canine teeth, can be whitened in an accurate and substantially uniform way. It is an advantage of embodiments according to the present invention that the tips of the canine teeth also are covered and therefore also can be whitened. It is an advantage of embodiments according to the present invention that good transport of the teeth bleaching moiety through the device can be obtained. It thereby is an advantage of embodiments according to the present invention that a folding line in the fibrous structure can be avoided, while still a good fit can be obtained and the folded shape over the teeth can be maintained during use of the device. Inhibition of the transport of teeth bleaching moiety or components thereof due to the presence of the folding line thus can be avoided. It is an advantage of embodiments of the present invention that the fibrous structure in the device provides the possibility of a good fit of the structure.

It is an advantage of embodiments according to the present invention that the fibrous structure of the device may allow water permeability, thus assisting in the uptake of teeth whitening moiety.

It is an advantage of embodiments according to the present invention that the fibrous structure may have a variable fibre diameter as function of the depth in the fibrous structure so as to control a profile in the release of teeth bleaching moiety.

It is an advantage of embodiments according to the present invention that methods and systems are provided allowing

The above objective is accomplished by a method and device according to the present invention.

The present invention relates to a teeth whitening system for whitening teeth, the teeth whitening system comprising a nano-fibrous structure and a teeth whitening moiety comprising a bleaching agent. It is an advantage of embodiments according to the present invention that methods and systems allow accurate delivery of teeth bleaching moiety or components thereof. It is an advantage of embodiments according to the present invention that methods and systems are provide allowing little or no leakage to the area of the mouth surrounding the teeth. It is an advantage of using a nano-fibrous structure that the system can adapt and maintain a good fit to the teeth.

The teeth whitening moiety may have a viscosity between 10 cps and 1000 cps, advantageously between 10 cps and 400 cps, more advantageously between 10 cps and 199 cps. It is an advantage of embodiments according to the present invention that methods and systems can be provided wherein the teeth whitening moiety may have relatively low viscosity, due to the fluid uptaking and releasing properties of the nano-fibrous structure. It is an advantage of embodiments according to the present invention that gels with low viscosity can be used for teeth whitening applications, whereby the gels can be kept in the system due to the nano-fibrous structure. It is an advantage of embodiments according to the present invention that the risk of leaking out of the structure, e.g. on the Gingiva or the Palate in the mouth is limited, reduced or even avoided.

The teeth whitening moiety may be a teeth whitening gel, wherein the teeth whitening gel furthermore may comprise a gel forming material. It is an advantage of embodiments according to the present invention that the use of gel reduces or prevents occurrence of leakage. The latter may increase the comfort for the user. It may for example result in a reduction of irritation of parts of the mouth surrounding the teeth.

The concentration of gel forming material may be lower than 0.1 weight percent of the teeth whitening moiety, e.g. lower than 0.09 weight percent of the teeth whitening moiety.

The gel forming material may comprise carboxymethylcellulose, carboxypropylcellulose, gum, poloxamer, or carboxypolymethylene. It is an advantage of embodiments according to the present invention that low concentrations of gel forming materials may be used as the viscosity of the bleaching moiety may be lower due to the presence of the nano-fibrous structure.

The nano-fibrous structure of the system may be adapted for fitting to a row of front teeth. It is an advantage of embodiments according to the present invention that the nano-fibrous structure is adapted so that it can be compressed to a set of teeth.

The nano-fibrous structure of the system may be adapted for fitting to incisor teeth and canine teeth, including the tips of the canine teeth. The incisor teeth are the four front teeth at the top or the four front teeth at the bottom of the mouth. It is an advantage of embodiments according to the present invention that the canine teeth adjacent to the incisor teeth also can be bleached, as these canine teeth often have a more yellowish colour and as bleaching also the canine teeth will result in a more homogeneous colour for canine and incisor teeth so that no large contrast between the canine teeth and the incisor teeth is obtained.

The nano-fibrous structure may be compressible to a thickness between 100 nm and 10 mm. It is an advantage of embodiments according to the present invention that a fitting over the incisor teeth as well as over the canine teeth can be made by using a compessible nano-fibrous structure that can fit to the teeth. It is an advantage of embodiments according to the present invention that the device, or at least the fibrous structure thereof, can be pressed to the teeth so that by compressing the device, or at least the fibrous structure thereof, the device or at least the fibrous structure thereof adopts to the shape of the teeth.

The nano-fibrous structure may have a thickness between 1 μm and 5000 μm, advantageously between 1 μm and 2000 μm.

The structure of the nano-fibrous structure may be adapted for inducing an adhesive effect of the system on the surface of teeth. It is an advantage of embodiments according to the present invention that no additional components or property requirements are to be posed on the teeth whitening moiety for having an adhesive effect on the teeth surface.

The nano-fibrous structure may have an average porosity between 65 and 99%, preferably between 70 and 98 and more preferably between 75 and 95%. It is an advantage of embodiments according to the present invention that the porosity of the nano-fibrous structure is adapted for inducing such adhesive effect.

The system may be adapted for being fixed at a front side of teeth using a first part and at a back side of the teeth using a second part.

The nano-fibrous structure may have a variation in porosity over its cross section adapted for providing a controlled transport of the bleaching agent towards the teeth.

The nano-fibrous structure may have a predetermined variation profile in porosity over its cross section adapted for obtaining a predetermined release profile of the bleaching agent towards the teeth. It is an advantage of embodiments according to the present invention that a controlled release of the bleaching agent can be provided, assisting in obtaining a homogeneous effect during the treatment.

The porosity of the nano-fibrous structure may increase from the side contacting the substrate layer to the side that will contact the surface of the teeth.

The nano-fibrous structure may be a laminated nano-fibrous structure comprising at least two layers of nanofibres having a different diameter.

The teeth whitening system may comprise a substrate layer for supporting the nano-fibrous structure, the substrate layer being substantially water impermeable and water insoluble. It is an advantage of embodiments according to the present invention that the substrate may provide a better fit of the system over the teeth. It is an advantage of embodiments according to the present invention that the substrate may provide a barrier for leakage of bleaching moiety e.g. to the gingival area and/or at the tongue.

The nano-fibrous structure comprises fibres comprising a pH setting agent. It is an advantage of embodiments according to the present invention that at least part of the pH setting agent can be kept separate from the bleaching agent before use or before preparation of the system for use. The latter is advantageous as it allows to increase the efficiency of the bleaching agent during use or it allows to increase the product lifetime of the system. It furthermore is an advantage of embodiments of the present invention that the pH-setting agent can be comprised in the nano-fibrous structure, avoiding the need for a further component to stored separately before use.

The teeth whitening moiety may comprise a pH setting agent.

The pH setting agent may comprise any or a combination of sodium hydroxide, hydrogen chloride, sodium phosphate, sodium bicarbonate, sodium stannate, citric acid or sodium citrate.

The pH setting agent in the moiety may be between 0.1 weight percent and 10 weigh percent.

The concentration of bleaching agent may be between 0.1 and 25 weight percent of the teeth whitening moiety, advantageously between 0.5 and 10 weight percent of the teeth whitening moiety, more advantageously between 1 and 7 weight percent of the teeth whitening moiety.

The bleaching agent may comprise any or a combination of peroxides or peroxide generating compounds. Peroxides may be for example hydrogen peroxide or calcium peroxide. Peroxide generating compounds may be percarbonates such as for example carbamide peroxide, perborates or peroxyacids. Bleaching materials that advantageously may be used are hydrogen peroxide or carbamide peroxide or a mixture thereof. It is an advantage of hydrogen peroxide that it has a high effectiveness.

The teeth whitening moiety furthermore may comprise a filling compound.

The filling compound may comprise one or more of glycerine, sorbitol, polyethylene glycol or propylene glycol.

At least 30%, preferably at least 50% of the nanofibres of the nano-fibrous structure may have an average diameter between 3 and 2000 nm.

The nano-fibrous structure may comprise at least 50% of straight fibres wherein the fibres have segments substantially straight over a distance of at least 5 μm.

The nano-fibrous structure may comprise at least 50% of randomly oriented fibres.

The nano-fibrous structure may be an electrospun nano-fibrous structure.

Upon application of the system between 2 and 30 minutes onto the teeth with a frequency of twice a day during a period of between 5 and 14 days, a teeth whitening benefit of at least 1 and maximum 14 shades on the vitashade scale may be obtained. It is an advantage of embodiments according to the present invention that these provide in good whitening effects.

The teeth whitening system may be a kit adapted to keep the nano-fibrous structure and the teeth bleaching moiety separate during storage before use.

The kit furthermore may comprise a tray for soaking the nano-fibrous structure in the teeth bleaching moiety when initiating use of the teeth whitening system.

The nano-fibrous structure may comprise fibres made of polyamide made by electrospinning using a mixture of formic acid and acetic acid. The ratio of formic acid and acetic acid may be between 90/10 and 10/90 weight percent, preferably between 30/70 and 70/30 weight percent and more preferably between 40/60 and 60/40 weight percent. It may be a 50/50 weight percent ratio.

The present invention also relates to the use of a teeth whitening system as described above for teeth bleaching.

The present invention also relates to a nano-fibrous structure, the nano-fibrous structure being adapted for use in a teeth whitening application.

The nano-fibrous structure may be adapted for fitting to a row of front teeth.

The nano-fibrous structure of the system may be adapted for fitting to incisor teeth and canine teeth, including the tips of the canine teeth.

The nano-fibrous structure may be compressible to a thickness between 100 nm and 5 mm.

The nano-fibrous structure may have a thickness between 1 μm and 5000 μm, advantageously between 1 μm and 2000 μm.

The structure of the nano-fibrous structure may be adapted for inducing an adhesive effect of the system on the surface of teeth.

The nano-fibrous structure may have an average porosity between 65 and 99%, preferably between 70 and 98 and more preferably between 75 and 95%.

The system may be adapted for being fixed at a front side of teeth using a first part and at a back side of the teeth using a second part.

The nano-fibrous structure may have a variation in porosity over its cross section adapted for providing a controlled transport of the bleaching agent towards the teeth.

The nano-fibrous structure may have a predetermined variation profile in porosity over its cross section adapted for obtaining a predetermined release profile of the bleaching agent towards the teeth.

The porosity of the nano-fibrous structure may increase from the side contacting the substrate layer to the side that will contact the surface of the teeth.

The nano-fibrous structure may be a laminated nano-fibrous structure comprising at least two layers of nanofibres having a different diameter.

The teeth whitening system may comprise a substrate layer for supporting the nano-fibrous structure, the substrate layer being substantially water impermeable and water insoluble.

The nano-fibrous structure may comprise fibres comprising a pH setting agent.

The pH setting agent may comprise any or a combination of sodium hydroxide, hydrogen chloride, sodium phosphate, sodium bicarbonate, sodium stannate, citric acid or sodium citrate.

The pH setting agent in the moiety may be between 0.1 weight percent and 10 weight percent of the moiety

The nano-fibrous structure may comprise fibres made of polyimide made by electrospinning using a mixture of formic acid and acetic acid. The ratio of formic acid and acetic acid between 90/10 and 10/90 weight percent, preferably between 30/70 and 70/30 weight percent and more preferably between 40/60 and 60/40 weight percent. It may be 50 weight percent to 50 weight percent.

The present invention also relates to the use of a nano-fibrous structure as described above for teeth bleaching.

The present invention also relates to a method for whitening teeth, the method comprising soaking a nano-fibrous structure in a teeth whitening moiety, applying the soaked nano-fibrous structure to the teeth for a predetermined time and removing the nano-fibrous structure thereafter. The teeth whitening moiety may be present in an initial container which may be sealed. The teeth whitening moiety may be packed together with the nano-fibrous structure. They may be packed in the container in which the teeth whitening moiety is to be poured. Such a package may be sealed with a plastic or cardboard film. For soaking the nano-fibrous structure, the nano-fibrous structure may be positioned in the container with the nano-fibrous structure facing the moiety.

The method furthermore may comprise providing initial contact between said pH regulating agent and said bleaching agent, during said soaking the nano-fibrous structure.

The method furthermore may comprise before said soaking the nano-fibrous structure, providing the teeth whitening moiety in a container adapted for soaking the nano-fibrous structure therein.

The present invention also relates to a method for manufacturing a nano-fibrous structure, the method comprising electrospinning nano-fibrous using a mixture of formic acid and acetic acid. The ratio of formic acid and acetic acid may be between 90/10 and 10/90 weight percent, preferably between 30/70 and 70/30 and more preferably between 40/60 and 60/40 weight percent. It may be 50 weight percent to 50 weight percent.

Particular and preferred aspects of the invention are set out in the accompanying independent and dependent claims. Features from the dependent claims may be combined with features of the independent claims and with features of other dependent claims as appropriate and not merely as explicitly set out in the claims.

Although there has been constant improvement, change and evolution of devices in this field, the present concepts are believed to represent substantial new and novel improvements, including departures from prior practices, resulting in the provision of more efficient, stable and reliable devices of this nature.

The teachings of the present invention permit the design of improved methods and apparatus for manufacturing fibrous structures with enhanced properties.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a side view of an electrospinning setup according to the prior art.

FIG. 2 is a schematic representation of a perspective view of an electrospinning device in operation according to an embodiment of the present invention.

FIG. 3 is a schematic representation of a perspective view of an electrospinning device in operation according to another embodiment of the present invention.

FIG. 4 is a schematic representation of a perspective view of jacketed tubes for temperature control of the solution or melt for use in embodiments of the present invention.

FIGS. 5A, B and C are schematic representations of a planar view of the positioning of the outlets for different embodiments for use in an electrospinning device according to embodiments of the present invention.

FIG. 6 is a magnified picture of a nanofibrous polymeric structure obtained in a comparative experiment.

FIG. 7 is a magnified picture of a nanofibrous polymeric structure according to an embodiment of the present invention.

FIG. 8 is a magnified picture of a nanofibrous polymeric structure obtained in a comparative experiment.

FIG. 9 is a magnified picture of a nanofibrous polymeric structure according to an embodiment of the present invention.

FIG. 10 is a magnified picture of a nanofibrous polymeric structure according to an embodiment of the present invention.

FIG. 11 is a magnified picture of a nanofibrous polymeric structure obtained in a comparative experiment.

FIG. 12 is a magnified picture of a nanofibrous polymeric structure according to an embodiment of the present invention.

FIG. 13 is a magnified picture of a nanofibrous polymeric structure obtained in a comparative experiment.

FIG. 14 is a magnified picture of a nanofibrous polymeric structure according to an embodiment of the present invention.

FIG. 15 is a magnified picture of a nanofibrous polymeric structure according to an embodiment of the present invention.

FIG. 16 is a magnified picture of a nanofibrous polymeric structure according to an embodiment of the present invention.

FIG. 17 is a table summarizing advantages of embodiments of the present invention.

FIG. 18 shows an example of a diagrammatic representation of a nano-fibrous structure which may be used in a teeth whitening system according to an embodiment of the present invention.

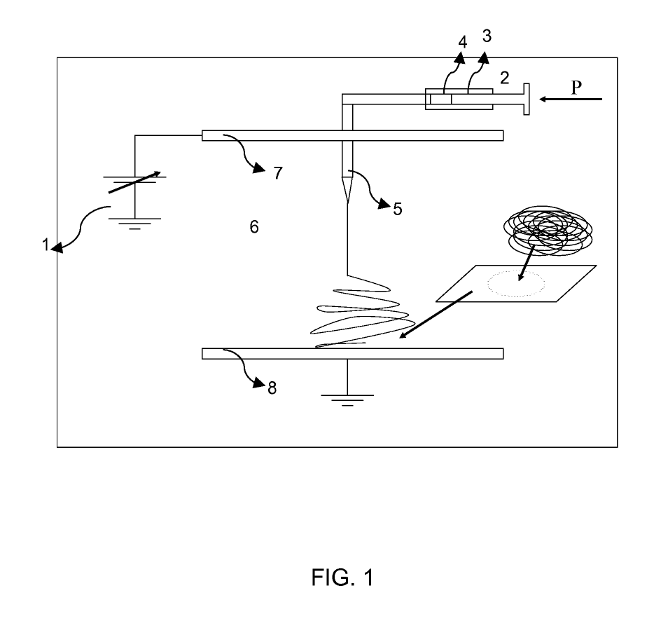

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

The invention will now be described by a detailed description of several embodiments of the invention. It is clear that other embodiments of the invention can be configured according to the knowledge of persons skilled in the art without departing from the technical teaching of the invention, the invention being limited only by the terms of the appended claims.

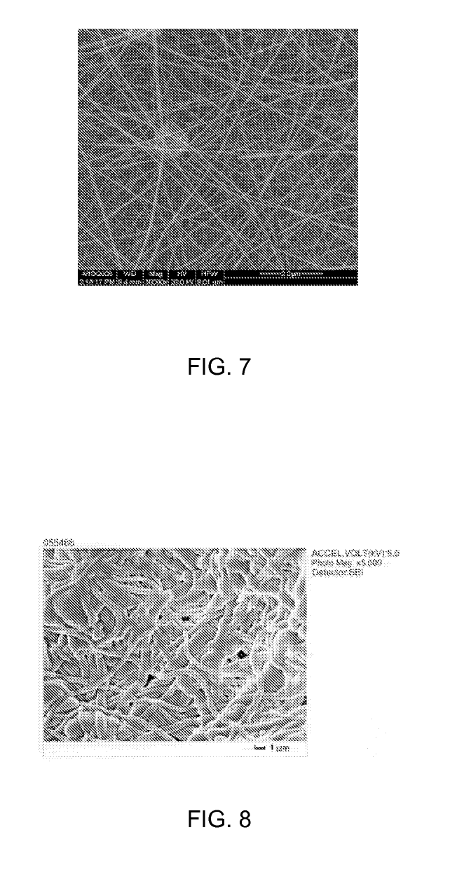

In a first aspect, the present invention relates to an electrospinning device for producing fibrous structures such as e.g. nanofibrous structures. In an embodiment of the first aspect, the electrospinning device comprises three or more outlets. The outlets may be of any nature known by the person skilled in the art to be suitable for electrospinning. The outlets are adapted for outputting material, e.g. solution or melt material to be used for the production of the fibers. The outlets may be of any nature known by the person skilled in the art to be suitable for electrospinning. For instance, the outlets may be nozzles, needles such as e.g. metalic needles, small holes or the likes. The electrospinning device may be a multi-nozzle device The three or more neighbouring outlets are separated from one another by a distance of at least 4 cm. For instance, the outlets may be separated by a distance of 4 to 100 cm. It has surprisingly been found that by separating the outlets by 4 cm or more the fibrous structures obtained were stronger, more porous and were comprising straighter fibers than for smaller spacing. The relatively large distance between the outlets (e.g. needles) allows an improved evaporation of the solvent, thus resulting in an improved porosity of the fibrous structure obtained. Without being bound by theory this effect may result from a more complete fiber formation process at the moment of collection of those fibers. Advantageously, the distance between each of the three or more outlets is at least 6 cm, more advantageously 8 cm or more. The maximum spacing is arbitrary and will for instance depend on the porosity one wishes to achieve. For a significant spacing between the outlets, the fibers constituting the fibrous structure acquire a surprising straightness over distances of 5 μm or more, 10 μm or more or even 20 μm or more as can be seen e.g. in FIG. 7 indicating the situation for a nozzle spacing of 6 cm. In parallel or in addition to this straightness, a majority of the fibres (i.e. 50% or more) constituting the fibrous structure becomes cross-link free, i.e. not cross-linked to neighboring fibers. The majority of the fibres is e.g. substantially cross-link free with respect to neighboring fibers at their contact points. According to embodiments of the present invention, devices are obtained that provide fibres that are cross-link free and thus not linked to each other, i.e. wherein the majority of the fibres, e.g at least 50%, advantageously at least 70%, more advantageously at least 90%, even more advantageously 95% remains independent. Cross link free thereby may be less than 1 cross link per 1 mm fiber length, advantageously less than 1 cross link per 5 mm fiber length, more advantageously less than 1 cross link per 1 cm fiber length, still more advantageously less than 1 cross link per 5 cm fiber length, even more advantageously without cross links over the full length of the fibre. This effect is particularly pronounced for outlets separated by 4 cm or more. Cross-link free thereby may be that there is absence of covalent bonds linking one polymer chain of one fibre to another polymer chain of a neighbouring fibre. The distance between the three or more outlets also may be adapted for obtaining a fibrous structure comprising at least 50% of fibres substantially free of any chemical bound. Weak physical interactions such as Van Der Walls interactions or hydrogen bridges are not covered by the definition of cross-links.

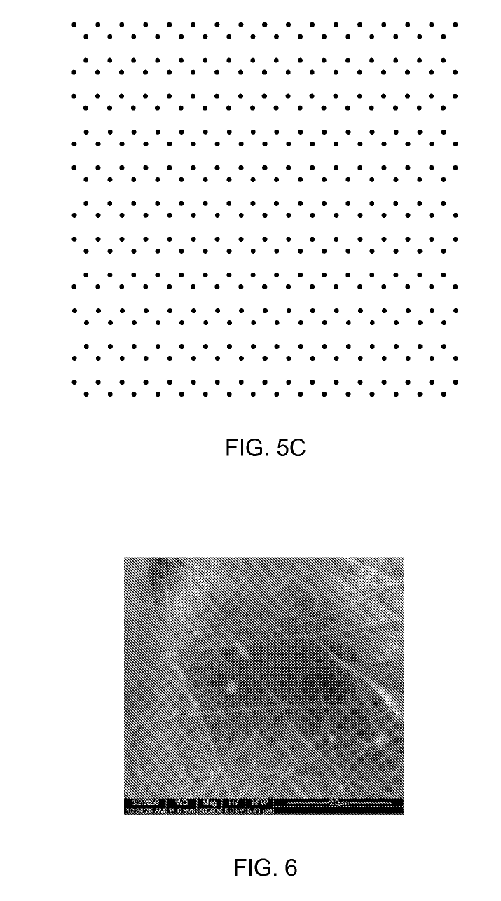

When three or more outlets are used, the outlets (e.g. needles) are advantageously arranged in sets of triangles with a distance between each outlet (e.g. needle) of minimum 4 cm and maximum 100 cm, more advantageously of minimum 6 cm and maximum 100 cm. The total number of outlets is not limited to a maximal value. For instance, the total number of outlets used in a configuration may be between 3 and 20000. Advantageously, the total number of outlets, e.g. needles, used in a configuration is between 3 (see FIG. 5A) and 500 (see FIG. 5B). In embodiments of the present invention where very volatile and/or easily ionizing solvents are used, the positioning of the needles may be adapted as shown in FIG. 5C. In FIG. 5C, the same individual positioning of the needles is respected as shown in FIG. 5B but for each two lines of outputs, e.g. needles, a third line is removed. In that case an individual output is never surrounded by needles at all sides. This permits an easier evaporation of the solvent from the fibre formation area. The purpose a needle set-up as shown in FIG. 5C is to avoid favouring electrical discharges when using volatile or ionizing solvents. The total number of outlets is not limited to a maximal value. For instance, the total number of outlets used in a configuration may be between 3 and 20000. It may be between 3 and 2000. Advantageously, the total number of outlets, e.g. needles, used in a configuration is between at least 3 and 500 (see FIG. 5). Different rows, such as e.g. neighbouring rows, of outlets may be parallel but shifted with respect to the corresponding position of the outlets with respect to each other. The latter may be evaluated with respect to the average direction of the relative movement of the second planar surface. The configuration of the outlets may be such that the outlets are positioned in triangular shaped groups of outlets. The different rows may for example result in a staggered configuration of outlets. The configuration may be such that for two neighbouring rows of outlets, a zigzag configuration of outlets is provided. The set of three or more outlets is positioned in a first plane, i.e. in a surface which may be not curved and not circular but has a planar shape. In other words, the different outlets have their output opening in the same plane. The surface defined by the plane is not necessarily solid or substantial, i.e. not necessarily comprised in a solid surface. In some embodiments, the first plane exist solely as a geometrical concept and the outlets are held in the first plane by e.g. a frame or any other structure capable of holding two or more outlets in a plane. In other embodiments, the surface and corresponding plane is material and forms a solid surface comprising the outlets.

The electrospinning device according to the present invention comprises also a second planar surface, which may be referred to as a receiving surface. The second planar surface is substantially parallel to the first plane and is facing the first plane. The second plane is a surface such as but not limited to a plate (e.g. a metallic plate), a foil or textile structure. The second planar surface may optionally be coated with a perforated or non-perforated layer, e.g. a perforated or non-perforated polymer/plastic layer. The second planar surface may be a planar part of a larger surface not necessarily planar in all its parts. The surface may contain a liquid surface on which the fibers are deposited. The second planar surface advantageously is parallel with the first plane. For instance, the second planar surface may be part of a larger belt comprising winded parts. The second planar surface is adapted for receiving output from the three or more outlets. The ensemble of the first plane and the second planar surface may take any spatial orientation. For instance, this ensemble may be horizontal with the first plane above the second planar surface or with the second planar surface above the first plane. In those cases, the outlets would therefore be oriented downward or upward respectively. For instance the outlets (e.g. needles) are positioned in a lower plate and solution or melt (e.g. polymer solution or melt) jets move upwards the device. In other embodiments, the ensemble of the first plane and the second planar surface is oriented vertically, Other orientations for the ensemble are of course possible (e.g. at 45° or any other angle with the horizon). The ensemble of, on one hand, the first plane comprising the outlets and on another hand the second planar surface is also referred to as a spinneret. At least one of the second planar surface and the set of outlets is adapted to be moveable, i.e. a relative movement may be provided between the second planar surface and the set of outlets. Preferably, at least one of these relative movements is in one direction parallel to the receiving surface and is responsible for the lengthwise growth of the fibrous structures. Preferably, this relative movement is caused by a movement of the receiving surface itself. The direction in which the set of outlets may be adapted to move can be either in the first plane or out of the first plane (e.g. perpendicularly to said first plane). The movement of the outlets can also be a combination of a movement in the first plane and out of the first plane. In embodiments of the present invention, the movement of the outlets is advantageously a reciprocal movement, e.g. a movement between two fixed points. The movement of the second planar surface may be parallel to said second planar surface, orthogonal to said second planar surface or a combination of both. Advantageously, the second planar surface can move continuously in one direction parallel to said second planar surface. Advantageously, the device is adapted for providing a relative movement to the set of outlets and the second planar surface, the relative movement being e.g. a combination of a relative movement in a first direction in the first plane and in a second direction parallel to the first plane and to the second planar surface but different from said first direction. For instance, the second planar surface can be adapted to undergo a relative movement at an angle to the first direction such as optionally substantially perpendicular to said first direction. Said first and second direction can be perpendicular to each other and parallel to said first plane and said second planar surface. In other embodiments, the first and second directions are perpendicular to each other and said first direction is perpendicular to said first plane and said second planar surface. Advantageously, the set of outlets and the second planar surface can move relatively to each other so that the set of outlets moves in one direction in the first plane (e.g. the x-direction), e.g. reciprocally such as e.g. between two inversion points, and the second planar surface moves continuously in a direction perpendicular to the first direction (e.g. the y-direction) but in the plane of said second planar surface. This type of reciprocal movement of the outlets is advantageous because it allows overlapping the output of the outlets as received on the second planar surface from the different outlets. The output of an outlet as received on the second planar surface may be referred to as the fibre umbrellas on the second planar surface. The fibre umbrellas have a high tendency to reject each other due to their charge and can never overlap if the configuration is used as a stationary system, i.e. if there is not at least a reciprocal relative movement between the second planar surface and the set of outlets. The amount of relative reciprocal movement may be selected such that the output of neighbouring outlets at least overlaps. The amount of relative reciprocal movement may be selected such that the output of neighbouring outlets at least overlaps. Additionally, the width of the obtained fibrous structure can be increased in this way, i.e. by using a reciprocal movement. The set of outlets is advantageously subject to a relative reciprocal movement with respect to the receiving surface with an average speed between 0.1 cm s⁻¹ and 100 cm s⁻¹ in the direction of the lengthwise growth of the fibrous structure. Further relative movement, preferably continuous relative movement between the outlets and the second planar surface allows continuous production of larger fibrous structure surface areas i.e. such a relative movement is responsible for the lengthwise growth of the fibrous structures. The relative reciprocal movement between the set of outlets is advantageously subject to a relative movement with respect to the second planar surface with an average speed between 0.1 cm s⁻¹ and 100 cm s⁻¹. The second planar surface is advantageously moveable with a speed between 10 cm h⁻¹ and 100 m h⁻¹. In some embodiments, the distance between the set of outlets and the second planar surface can be varied. For instance, the set of outlets may move perpendicularly (e.g. in the z direction) to said second planar surface. This enables to implement a fluctuation of the average fiber diameter as a function of thickness of the obtained fibrous structure. Similarly, the second planar surface may move perpendicularly (e.g. in the z direction) to said first plane. Only in those embodiments, i.e. when the distance between the set of outlets and the second planar surface can be varied, the number of outlets can be one or more instead of two or more

The electrospinning device of the present invention further comprises movement means for moving said set of outlets and/or said second planar surface, such as but not limited to one or more motors and one or more actuation means, such as e.g. transmission axis. The movement means may be adapted for inducing one or more of the relative movements as described above.

The electrospinning device of the present invention further comprises a voltage source adapted to apply a potential difference between the outlets and the second planar surface. The voltage source may be a DC-high voltage source able to apply a potential difference selected in the range between 100 and 200000 V over the spinneret, i.e. between the outlets and the second planar surface. For instance, the outlets (e.g. needles) may be electrically in contact with each other through a conductive (e.g. metallic) plate or holding structure. In other embodiments, a semi or non-conductive first material plane (e.g. a plate) or holding structure can be used in combination with means such as e.g. a metallic wire for electrically connecting all the outlets (e.g. needles). The voltage source may be connected to the first plane if this plane is substantive and electroconductive or to means (e.g. wire) for electrically connecting all the outlets (e.g. needles). The second planar surface is advantageously grounded. Optionally it can be used ungrounded (floating) but adapted security measures are then preferably taken.

The electrospinning device of embodiments of the present invention further may comprise at least one recipient for containing a solution or melt to be electrospun from said outlets. The recipient may contain a polymer solution or melt. Alternatively, the recipients may be external to the electrospinning device.

The electrospinning device of embodiments of the present invention advantageously further comprises means for providing the solution or melt to the outlets. The means for providing the solution or melt to the outlets can be any means known by the person skilled in the art. Examples of means for providing the solution or melt to the outlets comprise but are not limited to pumps or syringes among others as well as transfer means such as e.g. tubes.

For instance, each outlet (e.g. needle) can be fed with a solution or melt (e.g. a polymer solution or melt) by an individual means (such as e.g. an individual peristaltic pump). In some embodiments, a multichannel means (such as e.g. a peristaltic pump) can be used in which each channel feeds one individual outlet (e.g. needle). Also a multiple of multichannel means (e.g. pumps) can be used, dependent on the amount of outlets (e.g. needles) that need to be fed with polymer solution or melt. In other embodiments, an anesthesia type pump can be used to feed the outlets (e.g. needles) through syringes filled with polymer solution or melt and positioned in the anesthesia pump. In some embodiments, a multiple amount of outlets (e.g. needles) can be fed by one source being a peristaltic or anesthesia pump. The injection rate (e.g. the pump rate) of solution, e.g. polymer solution, or melt per outlet (e.g. needle) may be between 0.01 and 500 mL h⁻¹.

Solutions or melts usable within the present invention are any solution or melt known by the person skilled in the art to be suitable for forming fibers by electrospinning. The solution or melt can be obtained from polymers. Suitable polymers comprise but are not limited to polyamides, polystyrenes, polycaprolactones, polyacrylonitriles, polyethylene oxides, polylactic acids, polyacrylic acids, polyesteramides, polyvinyl alcohols, polyimides, polyurethanes, polyvinylpyrrolidon, collagen, cellulose and related products, chitosan, methacrylates, silk, polyethylene vinylacetate copolymer, polyehthylene vinylalcohol copolymer, polyvinylbutyral and combination thereof. The solution or melt may also contain metallic particles so that metal containing fibers can be formed.

As an optional feature, the solutions or melts may contain an additional compound, such as compounds with antibacterial, farmaceutical, hydrophobic/hydrophilic, anti corrosion, catalytic, oxidative/reductive and other properties.

The electrospinning device of the present invention may optionally further comprise a surrounding element, i.e. an element surrounding the other elements of the electrospinning device. For instance, the surrounding element can form a jacket around the spinneret and prevents the spinneret from instability such as air turbulence and/or allow solvent recuperation. Air turbulence are advantageously avoided in the spinneret because it may cause instability in the melt or solution jets and the fibre umbrellas produced by those jets on the second planar surface. The surrounding element may for instance be composed of plates of a non-conductive material connected to each other to form a closed embodiment, i.e. an enclosure.

The electrospinning device of the present invention may further comprise one or more optional temperature control means/systems. Those temperature control means may be added to the electrospinning device for instance in order to obtain higher reproducibility in fibre production. Fluctuations of temperature can have its influence on the evaporation rate of the solvent and thus on the final dimensions of the fibres and the porosity of the structures. Temperature controlling means are therefore advantageous. The solution or melt in the recipient may be temperature conditioned by using containers for (e.g. a liquid bath such as an oil or water bath) temperature control. The control of the temperature can also be operated during the solution transport from the recipient to the outlets via jacketed tubes that are connected directly or indirectly with a cooling/heating system such as said containers for temperature control. The spinneret may be temperature controlled by using means for bringing heated/cooled air in the spinneret. For instance, the electrospinning device of the present invention may comprises a temperature control system that allows to control the temperature in the range 280-1500 K.

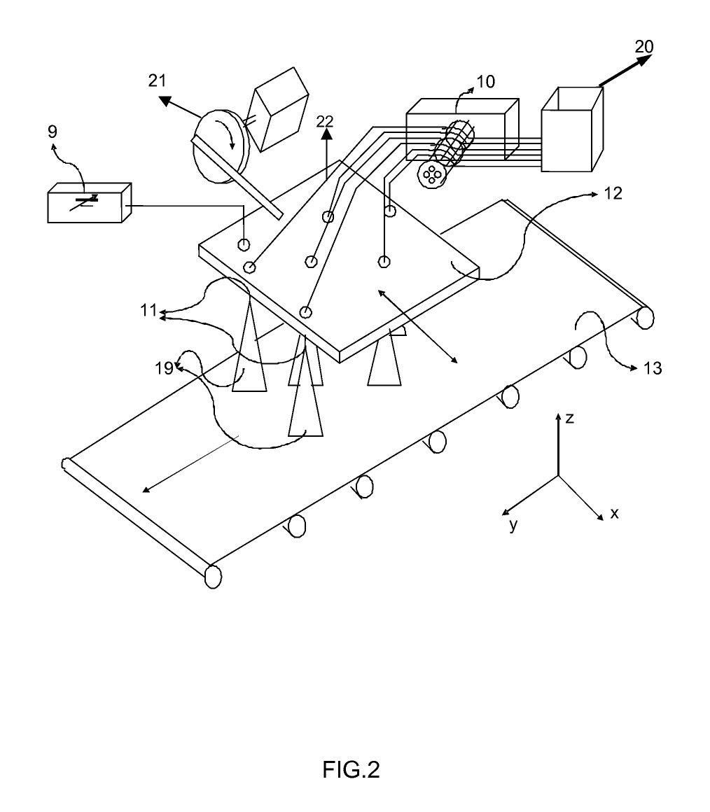

In FIG. 2, an electrospinning device according to one particular embodiment of the present invention is presented together with geometrical axes x, y and z. The z axis is the vertical axis while the x and the y axis defines two horizontal axis perpendicular to each other. This device comprises a high voltage source 9, a spinneret, said spinneret comprising a number of outlets 11 such as e.g. needles which are positioned in a first plane, e.g. comprised in a first planar plate 12, and a second planar surface 13. The system also comprises means 10 for providing a solution or melt to the outlets and means 21 for providing a relative movement of the set of outlets 11 with respect to the second planar surface 13. The device may comprise a recipient 20 and a transfer means 22 for providing solution or melt to the outlets.

In operation, the device depicted in FIG. 2 operates as follow: A voltage is set between the outlets 11 and the second planar surface 13. A liquid or melt to be electrospun is transferred from the recipient 20 to the outlets 11 via the transfer means 22 by the action of means 10 for providing a solution or melt to the outlets. The relative movement can be obtained by moving the outlets, e.g. by moving the first planar plate, reciprocally in the X direction by the operation of movement means 21 for moving the set of outlets. Simultaneously, the second planar surface may be moved continuously in the Y direction while collecting the fibrous structure formed by the overlap of the umbrellas caused by the melt or solution jets 19.

in FIG. 3, an electrospinning device according to another particular embodiment of the present invention is presented. It comprises all elements present in FIG. 2, and further comprises a means 18 for bringing heated/cooled air in the spinneret, a container for temperature control 17, and a surrounding element 14.

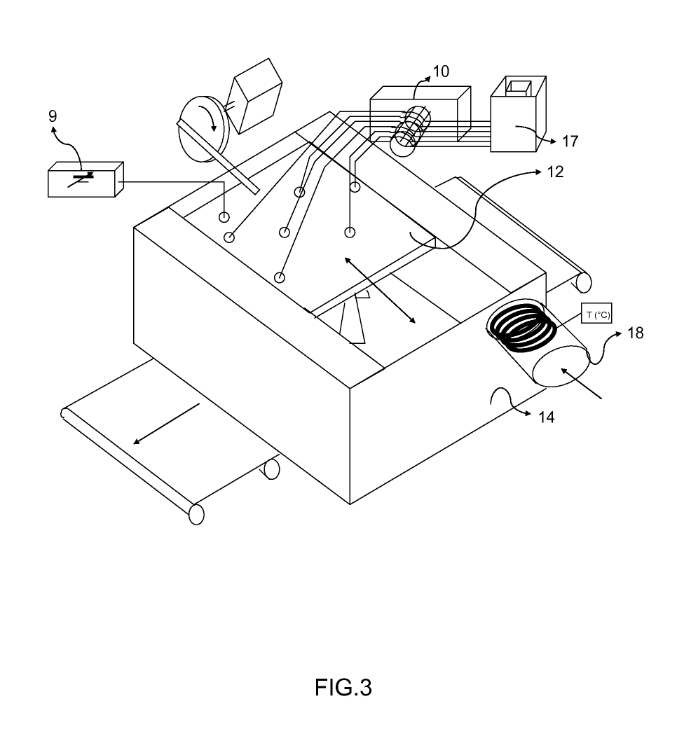

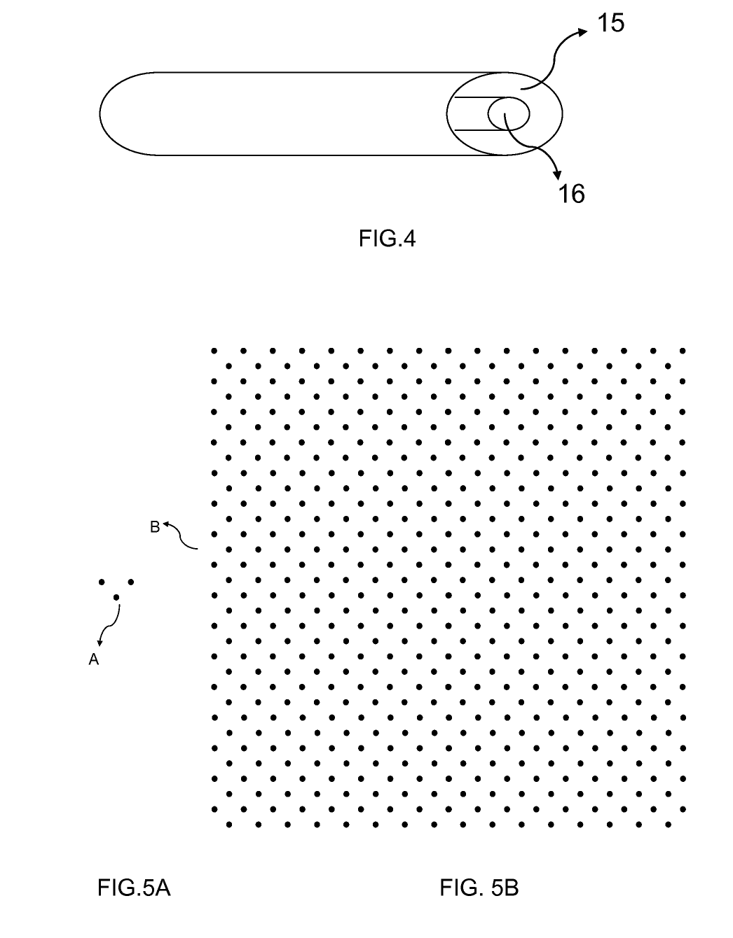

In FIG. 4, shows jacketed tubes comprising an inner tube 16 for solution or melt transport and a jacket 15 for liquid (e.g. oil) based temperature control.

According to the present aspect, the device furthermore may be adapted for generating a laminated fibrous structure, by altering the distance between the outlets and the second planar surface in a controlled way. The device therefore may comprise a controller for controlling movement of the outlets and the second planar surface during the production process of the fibrous structure. The controller may be adapted for selecting a first distance between the outlets and the second planar surface for obtaining a first layer of fibres and selecting one or more other distances between the outlets and the second planar structure for obtaining one or more further layers of fibres with different properties.

In a second aspect, the present invention relates to a method for producing fibrous structures. This method comprises the steps of applying a potential difference, i.e. a voltage between a set of three or more neighbouring outlets and a second planar surface, moving said set of outlets and said second planar surface relatively to each other, and providing a solution or melt to the outlets, wherein the outlets are separated from one another by a distance of at least 4 cm, The method may advantageously be performed with a system as described in the first aspect. The potential difference may be selected in the range between 100 and 200000 V. The movement step may be performed by actuating means for moving said set of outlets and/or said second planar surface. As a result, at least one of the second planar surface and the set of outlets is moved. The direction in which the set of outlets may move can be either in the first plane or out of the first plane (e.g. perpendicularly to said first plane). The movement of the outlets can also be a combination of a movement in the first plane and out of the first plane. The direction in which the set of outlets may further be moved can be parallel to the receiving surface, perpendicular to the receiving surface or a combination of both. The movement of the outlets may be a reciprocal movement, e.g. a movement between two fixed points preferably parallel to the receiving surface and perpendicular to the direction of the lengthwise growth of the fibrous structures. The direction in which the receiving surface may further move can be parallel to said receiving surface, orthogonal to said receiving surface or a combination of both. The movement of the second planar surface may be parallel to said second planar surface, orthogonal to said second planar surface or a combination of both. Advantageously, the second planar surface is moved continuously in one direction parallel to said second planar surface. Advantageously, the set of outlets is moved in a first direction in the first plane and the second planar surface is moved in a second direction parallel to the first plan and to the second planar surface but different from said first direction. For instance, the second planar surface can be moved at an angle to the first direction such as optionally substantially perpendicular to said first direction. Said first and second direction may be perpendicular to each other and parallel to said first plane and said second planar surface. Advantageously, the set of outlets and the second planar surface are moved relatively to each other so that the set of outlets moves in one direction in the first plane (e.g. the x-direction) between two inversion points and the second planar surface moves continuously in a direction perpendicular to the first direction (e.g. the y-direction) but in the plane of said second planar surface. The set of outlets may move with an average speed between OA cm s⁻¹ and 100 cm s⁻¹. The second planar surface may move with a speed between 10 cm h⁻¹ and 100 m h⁻¹.

The solution or melt may be kept in a recipient which may but does not have to be temperature controlled. Providing the solution or melt can be performed by solution or melt actuating means for providing the solution or melt to the outlets. Those means such as e.g. a pump transfer the solution or melt to the outlets via transfer means which may but do not have to be temperature controlled. Once at an outlets, the solution or melt forms a droplet from which a filament will be drawn and projected toward the second planar surface under the action of the potential difference. The second planar surface acts therefore as a collecting surface. The shape of the jet of solution or melt leaving an outlet is usually conical and forms a so-called umbrella, i.e. a covered area on the second planar surface. In an advantageous embodiment of the present invention, due to a reciprocal lateral relative movement of the outlets toward the receiving surface, the umbrella overlaps and form a fibrous structure such as a mat composed of fibres. The fibrous structures can in a later stage be recovered from the receiving surface by any method well known to the person skilled in the art. The fibrous structures can in a later stage be recovered from the second planar surface by any method well known to the person skilled in the art.

In embodiments of the second aspect, the present invention also relates to a method for producing fibrous structures having a porosity of at least 65%, said method comprising the steps of applying a potential difference between a first surface and a second surface, said first surface comprising three or more outlets, providing a relative movement between said first surface and said second surface resulting in movement of the first surface in a first direction while simultaneously moving said second surface in a second direction substantially perpendicular to said first direction, and providing a solution or melt to said outlets, wherein the three or more outlets are separated from one another by a distance of at least 1 cm.

In a third aspect, the present invention relates to a fibrous structure. The fibrous structure may for example comprising a majority, i.e. 50% or more of straight fibers. In one embodiment, the fibrous structure forms a mat. The method of the second aspect applied to the device of the first aspect permits to obtain fibrous structures having outstanding properties. A remarkable property being the straightness of the fibers comprised in the obtained fibrous structure. This straightness can be readily and directly observe in magnified pictures of the fibrous structures. This can for instance be observed in FIGS. 7, 9, 10, 12, 14, 15 and 16. An image analysis permits to determine that the majority of the fibers (i.e. 50% or more) comprised in the fibrous structure are straight, i.e. consists of a majority of segments (i.e. 50% or more) substantially straight over a distance of 5 μm. By substantially straight, it must be understood that the major axis of the fibre, i.e. along the direction of the fibre, changes over an angle less than 45°, e.g. less than 30°, or e.g. less than 15° or e.g. less than 5°, considering a distance of 10 micrometer over which the angle change was measured. This angle is the largest angle which can be measured between tangents at two points of the major axis over the length of the fiber considered. The standard deviation to linearity over the distance in question may be not exceeding 5%.

The electrospun fibrous structures of the present invention can be cut in any desired shape dependent on the requirements of the envisaged applications. The surface area can vary from 5 mm² to 10 m² and the thickness can vary from 100 nm to 30 cm.

The present invention also relates to a fibrous structure, e.g. microfibrous or nanofibrous structure wherein surprisingly a majority of the fibers (i.e. 50% or more) comprised are substantially cross-link free. The fibrous structure is an electrospun fibrous structure, it is a structure made by electrospinning. They are advantageously not cross-linked to neighboring fibers. Cross-linking thereby means that a link occurs between two fibres, not just that two fibers are touching. This is the result of the spacing between the outlets being at least 4 cm. Without being bound by theory, it is believed that this effect is due to an easier and therefore faster evaporation evaporation of the solvent during during the fibres formation. It is believed that for spacing between the outlets inferior to 4 cm, the solvent takes too much time to evaporate during the fibres formation. This leads to fusing of adjacent fibres and therefore to crosslinks. If the fibrous structure is made from a melt, the problem may be an incomplete elimination of the heating effect occurring when outlets are too close to each other. The fiber formation then is not complete and a sort of intermediate phase between melt and solid then may be present, allowing formation of cross-linked fibres. This effect is for instance clearly visible in FIGS. 6, 8, 11 and 13. The electrospun fibrous structures have preferably a porosity of at least 65%, advantageously between 65 and 99%. The pore sizes may vary from 30 nm to 8 μm. The present invention also relates to a fibrous structure wherein the thickness of the fibres is uniform, i.e. the standard deviation of the thickness throughout the fibrous structure does not exceed 80%, advantageously 50%, most advantageously 20%

The present invention also relates to a fibrous structure comprising more or all of the above identified properties. As an optional feature, the fibrous structures according to the present invention can be made comprising a majority of fibers (i.e. 50% or more) randomly oriented, i.e. not oriented in a particular direction (e.g. not aligned). The last effect is helpful in achieving an increased porosity. In embodiments where the second planar surface moves continuously in one direction, this effect can be obtained for example by choosing a speed for the second planar surface between 10 cm h⁻¹ and 100 m h⁻¹. As an optional feature, the diameter of a majority of the fibres (i.e. 50% or more of the fibers) comprised in the fibrous structures of the present invention have a diameter of 3 nm or higher, advantageously 10 nm or higher. As an optional feature, the diameter of a majority of the fibres (i.e. 50% or more) comprised in the fibrous structures of the present invention have a diameter of 2000 nm or lower, advantageously 800 nm or lower, most advantageously 700 nm or lower. When the fibers have a diameter with the range provided, i.e. between 3 nm and 2000 nm, they will be referred to as nanofibers and the fibrous structures made therefrom as nanofibrous structures. As an optional feature, the diameter of a majority of the fibers (i.e. 50% or more) comprised in the fibrous structures of the present invention have a diameter of 3 to 2000 nm, advantageously between 10 and 2000 nm, advantageously between 3 and 800 nm, more advantageously a diameter of 10 to 700 nm. As another optional feature, the fibrous structures obtained have a width between 15 and 10000 cm.

In embodiments of the third aspect of the present invention, the fibrous polymeric structures have a porosity of at least 65% and a width comprised between 15 and 10000 cm.

In some embodiments of the third aspect, the fibrous structures are obtained laminated, i.e. multi-layered. Advantageously, the average fibre diameter is different for each pair of adjacent layers within the fibrous structure. This may be achieved by using a different distance between the set of outlets and the second planar surface for each layer. The obtained laminated fibrous structures have a number of advantages compared to their non-laminated counter parts. Firstly, the combination of layers with small fiber diameter and layers with somewhat bigger fibers improve on the overall strength of the fibrous structure. Secondly, the absorption/release properties of the fibrous structure can be optimized as a function of application and this in a single production step and finally, multitasking and multi-functionality can be obtained by using a laminated structure, such as multilevel filtration in one single multilayered structure.

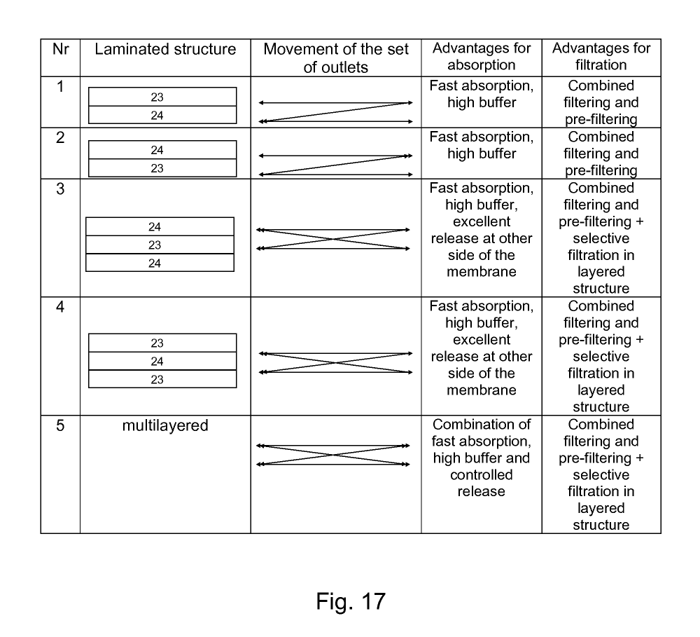

In FIG. 17, different ways are described to obtain laminated structures with minimum 2 layers. In column three is a visualization of the movement of the set of outlets to obtain the desired laminated structure (see FIG. 17).

In a first specific embodiment (FIG. 17, nr. 1), a fibrous structure with two layers 23 and 24 is obtained, each layer being comprised of fibres of different diameter. To obtain such a fibrous structure, first the set of outlets moves in y-direction from left to right and back with a preset frequency while the second planar surface is optionally moved continuously in the x-direction. After that the desired thickness of the first layer is obtained, the distance between the set of outlets and the second planar surface is adapted (e.g. reduced). That can be performed e.g. by moving from left to right (opposite is also possible) the set of outlets, while simultaneously moving the set of outlets downward in the z-direction. When it has reached its destination the plate continues to move from left to right in the lower position, resulting in the formation of a second layer with different diameters of the fibers. In a second specific embodiment (FIG. 17, nr. 2), which is similar to the first one, except that the two layers are inverted in relative position, the set of outlets moves from a lower to a higher position (instead of starting at the higher and moving to the lower) after that the first layer of fibers is formed. In a third specific embodiment (FIG. 17, nr. 3) a three layer structure is obtained by moving the set of outlets from left to right and back with a preset frequency, then going down to its second position where it continues to move between two points in y-direction. Finally, the set of outlets returns upward to its initial position and finishes the procedure again with a preset number of times moving between 2 points in the upper position. In yet another specific embodiment (FIG. 17, nr. 4) the set of outlets starts in its lower position, after formation of the first layer it moves to its upper position and finally returns to its initial lower position to produce the third layer. A high number of other embodiments is possible, e.g. with more than three layers, and by stacking layers of fibers, having more than two different average diameters of the individual fibers. These embodiments can be obtained by switching many times the position of the outlets in the z-direction and by using more than two positions at which the upper plate stays for formation of an individual layer of the fibrous structure.

### Example 1

Polyethylene oxide (PEO) with molecular weight of 300.000 g mol⁻¹ is dissolved in water to obtain a solution of 12% PEO. The solution is pumped to a set of needles with a multitude of multichannel peristaltic pumps, with a flow rate of 10 mL h⁻¹ per needle. In the spinneret an electrical field of about 800 V cm⁻¹ is applied over the upper and lower element in order to allow electrospinning of the polymer solution. Temperature control was performed at 298 K. In one setup 3 needles were used which were positioned at a distance of 2 mm (FIG. 6); in another setup the needles were positioned 6 cm from each other (FIG. 7). In the first setup the nanofibrous structures are less clear and have reduced porosity. With 3 needles a nanofibrous structure production rate of about 0.4 m h⁻¹ was obtained over a width of 60 cm, having a uniform thickness of 0.44±0.02 mm.

### Example 2

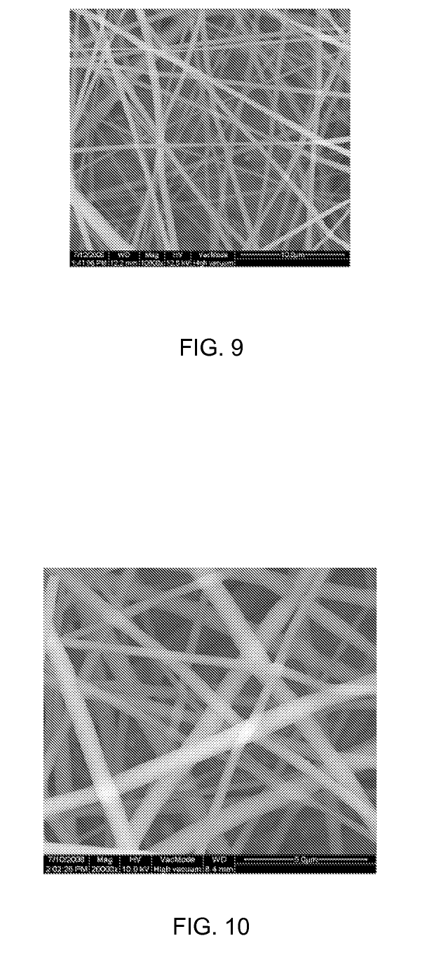

Polyester amide (PEA) with molecular weight of about 20.000 g mol⁻¹ is dissolved in chloroform to obtain a solution of 25% PEA. The solution is pumped to a set of needles with a multitude of multichannel peristaltic pumps, with a flow rate of 15 mL h⁻¹ per needle. In the spinneret an electrical field of about 1000 V cm⁻¹ is applied over the upper and lower element in order to allow electrospinning of the polymer solution. Temperature control was performed at 298 K. In one setup 3 needles were used which were positioned at a distance of 2 mm (FIG. 8); in another setup the needles were positioned 6 cm from each other (FIG. 9). With 3 needles a nanofibrous structure production rate of about 0.5 m h⁻¹ was obtained over a width of 60 cm, having a uniform thickness of 0.50±0.02 mm.

### Example 3

Cellulose acetate (CA) with molecular weight of 30.000 g mol⁻¹ is dissolved in cyclohexanol to obtain a solution of 8% CA. The solution is pumped to a set of needles with a multitude of multichannel peristaltic pumps, with a flow rate of 10 mL h⁻¹ per needle. In the spinneret an electrical field of about 1100 V cm⁻¹ is applied over the upper and lower element in order to allow electrospinning of the polymer solution. Temperature control was performed at 298 K. In one setup 3 needles were used which were positioned at a distance of 2 mm; in another setup the needles were positioned 6 cm from each other (FIG. 10). For the first setup no nanofibrous structure was obtained. With 3 needles a nanofibrous structure production rate of about 0.3 m h⁻¹ was obtained over a width of 60 cm, having a uniform thickness of 0.40±0.02 mm.

### Example 4

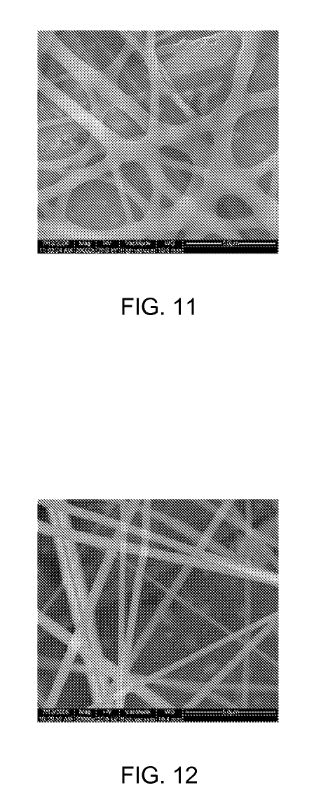

CA with molecular weight of 30.000 is dissolved in Aceton/EtOH to obtain a solution of 12% CA. The solution is pumped to a set of needles with a multitude of multichannel peristaltic pumps, with a flow rate of 6 mL h⁻¹ per needle. In the spinneret an electrical field of about 700 V cm⁻¹ is applied over the upper and lower element in order to allow electrospinning of the polymer solution. Temperature control was performed at 298 K. In one setup 3 needles were used which were positioned at a distance of 2 mm (FIG. 11); in another setup the needles were positioned 6 cm from each other (FIG. 12). With 3 needles a nanofibrous structure production rate of about 0.3 m h⁻¹ was obtained over a width of 60 cm, having a uniform thickness of 0.40±0.02 mm.

### Example 5

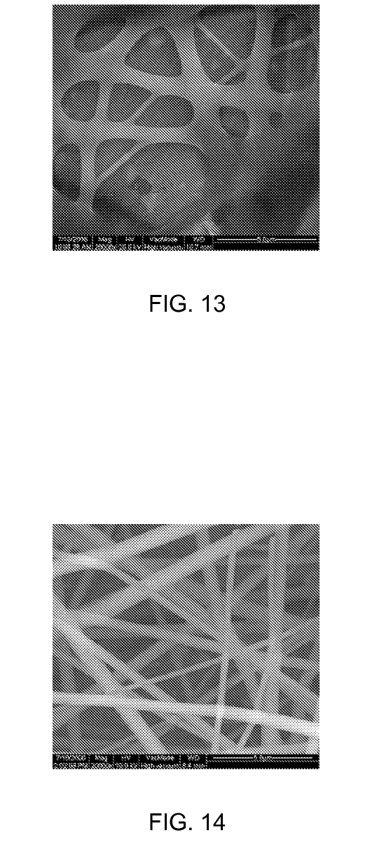

CA with molecular weight of 40.000 is dissolved in Aceton/DMA to obtain a solution of 12% CA. The solution is pumped to a set of needles with a multitude of multichannel peristaltic pumps, with a flow rate of 10 mL h⁻¹ per needle. In the spinneret an electrical field of about 800 V cm⁻¹ is applied over the upper and lower element in order to allow electrospinning of the polymer solution. Temperature control was performed at 298 K. In one setup 3 needles were used which were positioned at a distance of 2 mm (FIG. 13); in another setup the needles were positioned 6 cm from each other (FIG. 14). With 3 needles a nanofibrous structure production rate of about 0.18 m h⁻¹ was obtained over a width of 60 cm, having a uniform thickness of 0.30±0.02 mm.

### Example 6

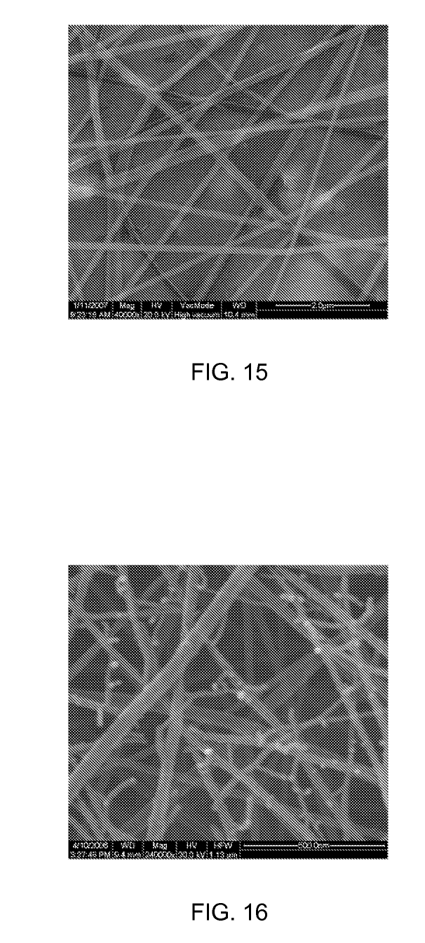

Polyamide 6/6 (PA66) is dissolved in formic acid to obtain a solution of 14% PA66. The solution is pumped to a set of needles with a multitude of multichannel peristaltic pumps, with a flow rate of 5 mL h⁻¹ per needle. In the spinneret an electrical field of about 4800 V cm⁻¹ is applied over the upper and lower element in order to allow electrospinning of the polymer solution. Temperature control was performed at 298 K. In one setup 3 needles were used which were positioned at a distance of 2 mm; in another setup the needles were positioned 6 cm from each other (FIG. 15). In the first setup a solid structures instead of nanofibrous structure was obtained. With 3 needles a nanofibrous structure production rate of about 0.3 m h⁻¹ was obtained over a width of 60 cm, having a uniform thickness of 0.40±0.02 mm.

### Example 7

Chitosan with molecular weight of about 250.000 g mol⁻¹ is dissolved in acetic acid (90%) to obtain a solution of 3% Chitosan. The solution is pumped to a set of needles with a multitude of multichannel peristaltic pumps, with a speed of 0.5 mL h⁻¹ per needle. In the spinneret an electrical field of about 2000 V cm⁻¹ is applied over the upper and lower element in order to allow electrospinning of the polymer solution. Temperature control was performed at 298 K. In one setup 3 needles were used which were positioned at a distance of 2 mm; in another setup the needles were positioned 6 cm from each other (FIG. 16). For the first setup no nanofibrous structure was obtained. With 3 needles a nanofibrous structure production rate of about 0.01 m h⁻¹ was obtained over a width of 60 cm, having a uniform thickness of 0.30±0.02 mm.

### Example 8

TiO₂ nanofibrous structures were obtained from dissolving Ti-isopropoxide and polyvinylpyrrolidon with molecular weight of 900.000 g mol⁻¹ in EtOH to obtain a solution. The solution is pumped to a set of needles with a multitude of multichannel peristaltic pumps, with a speed of 16 mL h⁻¹ per needle. In the spinneret an electrical field of about 1800 V cm⁻¹ is applied over the upper and lower element in order to allow electrospinning of the polymer solution. Temperature control was performed at 298 K. In one setup 3 needles were used which were positioned at a distance of 2 mm; in another setup the needles were positioned 6 cm from each other. With 3 needles a nanofibrous structure production rate of about 0.1 m h⁻¹ was obtained over a width of 60 cm, having a uniform thickness of 0.40±0.02 mm.

### Example 9

PA66 is dissolved in formic acid/acetic acid to obtain a solution of 15% PA66. The solution is pumped to a set of needles with a multitude of multichannel peristaltic pumps, with a speed of 6 mL h⁻¹ per needle. In the spinneret an electrical field of about 5000 V cm⁻¹ is applied over the upper and lower element in order to allow electrospinning of the polymer solution. Temperature control was performed at 298 K. In one setup 3 needles were used which were positioned at a distance of 2 mm; in another setup the needles were positioned 6 cm from each other. Similar pictures were obtained as in example 6. With 3 needles a nanofibrous structure production rate of about 0.3 m h⁻¹ was obtained over a width of 60 cm, having a uniform thickness of 0.44±0.02 mm.

The present invention also relates to nanofibrous structures for dental application, more particularly for teeth bleaching.

According to a fourth aspect, the present invention relates to a teeth whitening system for whitening teeth. The system for whitening teeth comprises two parts. A first part comprises a nano-fibrous structure. A second part comprises a teeth whitening moiety comprising a bleaching agent. The nano-fibrous structure may be used for keeping the teeth whitening moiety during use of the system for bleaching teeth. In other words, the nano-fibrous structure may act as a carrier for the teeth whitening moiety during use. The nano-fibrous structure may be provided to a plurality of teeth for teeth whitening, also referred to as teeth bleaching. The structure of the nano-fibrous structure may be adapted so that it can appropriately absorb, keep and release moiety with a viscosity between 10 cps and 1000 cps, advantageously between 10 cps and 400 cps, more advantageously between 10 cps and 199 cps.

The nano-fibrous structure may be supported by a substrate layer, although the invention is not limited thereto. The optional substrate layer can be any material that is certified for use in the mouth. It may for example be a material that can adopt to the shape of the teeth such as aluminium foil because it is suitable for nano-fibrous deposition in the electrospinning method and it keeps its shape after bending over the teeth. Nevertheless, the nano-fibrous structure also may itself be adapted in structure so that it can keep its shape when provided on the teeth.

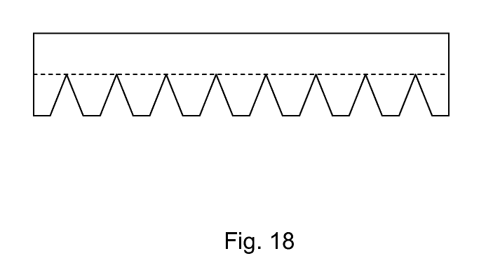

The nano-fibrous structure may be adapted for fitting to a plurality of teeth, e.g. a row of front teeth. The nano-fibrous structure may be responsible for the adhesive effect between this layer and the surface of the teeth based on its high contact surface area. The nano-fibrous structure may be adapted in structure for having an adhesive effect of the device on the teeth. The teeth to be treated may comprise incisor teeth. Advantageously, the structure is adapted for treating the incisor teeth as well as the canine teeth simultaneously. In an advantageous embodiment, the structure may be adapted for covering the tips of the canine teeth, e.g. in combination with a coverage of the incisor teeth. The nano-fibrous structure may be adapted for being fixed at a front side of the teeth using a first part and for being fixed to a back side of the teeth using a second part. The drawings in FIG. 18 refer to an exemplary first part of the system composed of an optional substrate coated with a nano-fibrous structure, e.g. a nano-fibrous structure. At one side of the first part of the device triangles are cut out to allow bending of the first part of the device and fit the areas that are left to the back side of the teeth. The full area of the first part of the device is brought onto the front surface of the teeth. Initially, the first part of the device is flat but when applied to the teeth it bends and fits perfectly to the shape of the teeth. The feature to bend it over the teeth and to allow a triangle shape being fit at the backside, a perfect fit with the teeth shape is finally obtained through the fact that the nanofiber layer is a very porous, compressible and fluffy structure, thus surrounds perfectly around the shape of each individual tooth.

The device has a length between 0.5 and 15.0 cm, more preferably between 2 and 10 cm and most preferably between 4 and 8 cm and a width between 0.3 and 5.0 cm, more preferably between 0.5 and 4 cm and most preferably between 1 and 3 cm. At one length side of the first part of the device triangles are cut out in order to allow easy bending and fitting of the first part of the device over the teeth. These triangles penetrate in the first part of the device with a deepness between 0.1 and 2.0 cm, more preferably between 0.3 and 1.5 and most preferably between 0.5 and 1 cm and have legs with a length between 0.13 and 2.5 cm, more preferably between 0.4 and 2.0 cm and most preferably between 0.7 and 1.3 cm. In an alternative embodiment the edges of the strip are rounded to avoid possible injuries in the mouth. Other suitable shapes for the device also may be provided.

The nano-fibrous structure may comprise nanofibres such that at least 30%, advantageously at least 50% of the nanofibres of the nano-fibrous structure have an average diameter between 3 and 2000 nm. The nano-fibrous textile structure, deposited at the substrate, may have an average thickness between 1 μm and 5000 μm, advantageously between 1 μm and 2000 μm, advantageously between 10 and 2000 nm, advantageously between 3 and 800 nm, more advantageously a diameter of 10 to 700 nm. The diameter of a majority of the fibres (i.e. 50% or more of the fibers) comprised in the fibrous structures of the present invention may have a diameter of 3 nm or higher, advantageously 10 nm or higher. As an optional feature, the diameter of a majority of the fibres (i.e. 50% or more) comprised in the fibrous structures of the present invention have a diameter of 2000 nm or lower, advantageously 800 nm or lower, most advantageously 700 nm or lower. For the present application, fibres with diameters with one of the suggested diameter or within one of the diameter ranges will be referred to as nanofibres, and the corresponding structures may be referred to as nano-fibrous structures. In embodiments, the present invention also relates to a fibrous structure wherein the thickness of the fibres is uniform, i.e. the standard deviation of the thickness throughout the fibrous structure does not exceed 80%, advantageously 50%, more advantageously 20%. The fibre diameter is dependent on the distance between the outlets and the receiving surface. The profile of the relationship between the fibre diameter and the distance may be polymer and solvent specific. Therefore a profile can be determined after studying the polymer solution or melt because it is polymer and solvent specific. It can be determined via trial and error, via experimental results, via a theoretical model, etc. The individual nanofibres may have a length between 10 μm and 50 m.

The fibre material can be any suitable material, such as for example polyamides, polystyrenes, polycaprolactones, polyacrylonitriles, polyethylene oxides, polylactic acids, polyacrylic acids, polyesteramides, polyvinyl alcohols, polyimides, polyurethanes, collagen, cellulose and related products, chitosan, methacrylates, silk and metal containing nanofibers. The nano-fibrous structure also may comprise fibres comprising a pH regulating material, as will be described further in the application. Other components of the device also could be introduced in the nano-fibrous structures, e.g. by spinning particular nano-fibres thereof.

The structure may have a porosity, e.g. an average porosity, of at least 65%. The porosity, e.g. average porosity, may be between 65 and 99%, advantageously between 70 and 98 and more advantageously between 75 and 95%. The pore sizes can vary from 30 nm to 8 μm. The nano-fibrous structure may for example be able to absorb water, a solution, a compound or a gel for an equivalent of 3 to 12 times its own weight.

The nano-fibrous structure may comprise at least 50% of straight fibres wherein the fibres have segments substantially straight over a distance of at least 5 μm. The straightness of the fibres can for instance be inferred from an image analysis. Preferably, the majority of the fibers (i.e. 50% or more) comprised in the fibrous structure are straight, i.e. consists of a majority of segments (i.e. 50% or more) substantially straight over a distance of 5 μm. By substantially straight, it must be understood that the major axis of the fibre, i.e. along the direction of the fibre, changes over an angle less than 45°, e.g. less than 30°, or e.g. less than 15° or e.g. less than 5°, considering a distance of 10 micrometer over which the angle change was measured. This angle is the largest angle which can be measured between tangents at two points of the major axis over the length of the fiber considered. The standard deviation to linearity over the distance in question may be not exceeding 5%.

As an advantageous feature, the fibrous structures of the present invention may comprise only few or no crosslinking, e.g. microfibrous or nano-fibrous structure wherein a majority of the fibers (i.e. 50% or more) comprised are substantially cross-link free. They are advantageously not cross-linked to neighboring fibers. Cross-linking thereby means that a link occurs between two fibres, not just that two fibers are touching. This is the result of the spacing between the outlets being at least 4 cm. Without being bound by theory, it is believed that this effect is due to an easier and therefore faster evaporation of the solvent during the fibres formation. It is believed that for spacing between the outlets inferior to 4 cm, the solvent takes too much time to evaporate during the fibres formation. This leads to fusing of adjacent fibres and therefore to crosslinks. If the fibrous structure is made from a melt, the problem may be an incomplete elimination of the heating effect occurring when outlets are too close to each other. The fiber formation then is not complete and a sort of intermediate phase between melt and solid then may be present, allowing formation of cross-linked fibres.

The nano-fibrous structure may comprise at least 50% of randomly oriented fibres. In one embodiment, the fibrous structure forms a mat.

Advantageously the nano-fibrous structure may be obtained through electrospinning of a material. Fibres with the features as indicated may be obtained by an electrospinning technique with a multi-nozzle system. The nozzles may be separated at an interdistance of at least 1 cm, e.g at least 4 cm. Furthermore, for controlling the diameter, the distance between the nozzles and the receiving surface may be adapted. Varying the distance during electrospinning may allow obtaining a variable diameter of the fibres in the nano-fibrous structure. Variation of other parameters for electrospinning in view of the variation of the outlet collector distance, such as for example the voltage to be applied, may for example be determined experimentally.

In particular embodiments according to the present invention, the nano-fibrous structure may have a variation in porosity, diameter of the fibres or density of the fibres in one or mare dimensions, such as for example in the depth direction of the nano-fibrous structure, i.e. over the cross-section, and/or in the surface direction of the nano-fibrous structure, i.e. in a plane along the surface of the nano-fibrous structure which is suitable for contacting with the teeth. The variation of the porosity, diameter of the fibres or density of the fibres may be according to a predetermined variation profile in order to provide a predetermined release profile of the teeth whitening moiety or more particularly a bleaching agent thereof.

In one example, the porosity of the nano-fibrous structure may increase from the side contacting the substrate layer to the side that will contact the surface of the teeth. Variation in porosity, diameter of the fibres or density of the fibres may be obtained continuously or stepwise. In one example, such a variation may be obtained by using a laminated nano-fibrous structure comprising at least two layers of nanofibres having a different diameter.

Providing a predetermined profile in density, porosity or diameter thus may provide a predetermined rate of release of the teeth bleaching agent, which may be advantageous for providing a treatment according to a predetermined profile, e.g. with a uniform bleaching agent release. Other profiles also may be provided. For example, release of a large portion, e.g. about 40% to 50% of the bleaching agent may be provided during an initial period, e.g. the first 4 minutes, while the remaining portion may be released over a larger period of time, e.g. the following 9 minutes, in a subsequent period. In one aspect, the present invention also relates to a nano-fibrous structure as described in embodiments of the present invention adapted for use in a teeth whitening application. The same features and advantages related to the nano-fibrous structure as described in these embodiments also apply to this aspect of the invention.

As indicated above, the teeth whitening moiety comprises a bleaching agent, which may be the active component for bleaching a tooth or a plurality of teeth or an agent comprising such a component or able to release or generate such a component. The bleaching agent, sometimes also referred to as bleaching compound, may be a peroxide, such as for example hydrogen peroxide or a hydrogen peroxide generating product, calcium peroxide or a calcium peroxide or a combination thereof. The bleaching agent also may be a peroxide generating compound such as carbamide peroxide, perborates, percarbonates, oxyacids, an/or combinations of these chemicals. For example, the bleaching component concentration, e.g. hydrogen peroxide concentration that may be used may be between 0.1 and 35 weight percent of the teeth whitening moiety, preferably between 1 and 25 weight percent of the teeth whitening moiety and more preferably between 3 and 16 weight percent of the teeth whitening moiety. The concentrations of the bleaching agent, e.g. hydrogen peroxide, may be adopted in that way that the concentration of active component, e.g. the hydrogen peroxide, may be between 0.1 weight percent and 35 weight percent, preferably between 1 weight percent and 25 weight percent and more preferably between 3 weight percent and 16 weight percent.

The teeth whitening moiety may comprise a filling compound. The filling compound may comprise one or more of glycerine, sorbitol, polyethylene glycol or propylene glycol.

The teeth bleaching structure also may comprise a pH regulation compound, which is a regular and state of the art standard buffer. It may for example be any or a combination of sodium hydroxide, hydrogen chloride, sodium phosphate, sodium bicarbonate, sodium stannate, citric acid or sodium citrate, the invention not being limited thereto. The pH setting agent in the moiety may be between 0.1 weight percent and 10 weight percent. In some embodiments, the pH regulating agent may be provided directly in the teeth bleaching moiety. As the activity, the stability and the decomposition of the bleaching agent often depends on the pH of the moiety in which it is present, a pH regulating agent may be added to have an optimized activity of the bleaching agent. Nevertheless, providing a pH regulating agent immediately in the teeth bleaching moiety results the need for a trade-off between little activity of the bleaching agent during storage and sufficient activity of the bleaching agent during use. Such a trade-off nevertheless limits the on-the-shelve lifetime of the teeth bleaching product, or at least the lifetime during which the system is most efficient.

According to preferred embodiments of the present invention separate delivery of the pH regulating agent and the bleaching agent may be provided. For storage of the bleaching agent, the pH of the moiety wherein the bleaching agent is stored is chosen so as to have a low decomposition rate, such as e.g. in a moiety with pH of about 5, so that the bleaching agent is quite stable. Upon mixing with the pH regulating agent, when preparing for use or using, the pH of the moiety can shifts to 7.5-8.0 due to the functioning of the pH regulating agent. The latter may for example occur when the teeth whitening agent' is taken up by the nano-fibrous structure to or in which the pH regulating agent is provided. At this pH the bleaching agent is less stable but also more active in bleaching. Therefore it is advantageous to use a pH around 7.5 for bleaching but a pH clearly below 6 for storage.

One particular solution to overcome the storage problem is provided in one embodiment, where the pH regulating agent is applied as a powder on the nano-fibrous structure or on the substrate carrying the nano-fibrous structure if present. Alternatively or in addition thereto the bleaching agent may be provided separately to the nano-fibrous structure or the substrate supporting it. In a more preferred embodiment the pH regulating agent is provided in the fibres of the nano-fibrous structure. The latter may for example be obtained by adding the appropriate amount of pH regulating material to the solution for making the nano-fibrous structure, e.g. the electrospinning solution. The latter has the advantage that the different actions needed to be performed by the user are limited and that the different components that need to be stored separately before use or preparation thereof can be limited or reduced.

The teeth whitening moiety may be a gel. It may comprise a gel forming material. The gel forming material may have a concentration lower than 0.1 weight percent of the teeth whitening moiety, e.g. lower than 0.09 weight percent of the teeth whitening moiety. The gel forming material may comprise any or a combination of carboxymethylcellulose, carboxypropylcellulose, gum, poloxamer, or carboxypolymethylene. It is an advantage of embodiments according to the present invention that the use of gel reduces or prevents occurrence of leakage. The latter may increase the comfort for the user. It may for example result in a reduction of irritation of parts of the mouth surrounding the teeth.

The teeth whitening moiety may comprise a taste product, in order to promote a good taste when teeth bleaching is performed. Examples of such taste products may for example be spearmint/peppermint.

It is an advantage of embodiments according to the present invention that the viscosity of the teeth whitening moiety may be relatively low. The viscosity may be between 10 and 1000 cps, advantageously between 10 cps and 400 cps, more advantageously between 10 cps and 199 cps. It is an advantage of embodiments according to the present invention that methods and systems can be provided wherein the teeth whitening moiety may have relatively low viscosity, due to the fluid uptaking and releasing properties of the nano-fibrous structure. It is an advantage of embodiments according to the present invention that the risk of leaking out of the structure, e.g. on the Gingiva or the Palate in the mouth is limited, reduced or even avoided. Alternatively not a gel but an aqueous solution can be used and immobilized in the nano-fibrous structure. Additionally, a low viscosity results in higher mobility of the bleaching compound in the gel, thus release of bleaching agent to the teeth is more efficient.

The teeth whitening system may comprise a Gingiva or Palate protecting component, such as for example a chemical component for protecting the Gingiva or Palate protecting component like GANTREZ®, the present invention not being limited thereto.

According to an embodiment of the present invention, the teeth whitening system may be a kit adapted to keep the nano-fibrous structure and the teeth bleaching moiety separate during storage before use. When the pH regulating agent is provided in the nano-fibrous structure, or at least separate from the teeth bleaching agent, the latter results in the possibility to operate the bleaching at an optimised pH resulting in a high efficiency of the bleaching agent. The teeth whitening system may be packed in a blister which itself may be used as a soaking tray for soaking the nano-fibrous structure in the teeth whitening moiety. The device thus may be delivered to the user in two parts wherein the nano-fibrous structure containing the pH regulator is one part and the moiety is another part.

In still another aspect, the present invention relates to a method for performing teeth bleaching. The method according to embodiments of the present invention may comprise using both a nano-fibrous structure and a teeth whitening moiety for performing the teeth bleaching. The method may be applied in any suitable place, e.g. at the consumer's choice, i.e. not being limited to the dental practice but for example also at home. The method has the advantage that it provides bleaching of teeth, which results in an aesthetic effect. The method may be performed using a teeth bleaching system as described in any of the embodiments according to the fourth aspect of the present invention, although the invention is not limited thereto. The teeth whitening moiety may be packed together with the nano-fibrous structure in one overall package but the teeth whitening moiety advantageously is not in direct contact with the nano-fibrous structure during storage. The nano-fibrous structure and/or the teeth whitening moiety may be packed in the container in which the teeth whitening moiety is to be poured. Such a package may be sealed with a plastic or cardboard film. The teeth whitening moiety may be present in an initial container which may be sealed. The method may comprise before said soaking the nano-fibrous structure, providing the teeth whitening moiety in a container adapted for soaking the nano-fibrous structure therein.

The method according to embodiments of the present invention, comprises soaking a nano-fibrous structure in a teeth whitening moiety. For soaking the nano-fibrous structure, the nano-fibrous structure may be positioned in the container with the nano-fibrous structure facing the moiety. The method furthermore may comprise providing initial contact between said pH regulating agent and said bleaching agent, during said soaking the nano-fibrous structure.

According to embodiments of the present invention, the method also comprises applying the soaked nano-fibrous structure to the teeth for a predetermined time and removing the nano-fibrous structure thereafter. Applying the soaked nano-fibrous structure may comprise adapting the shape of the nano-fibrous structure to the shape of the teeth. By using a nano-fibrous structure, the shape of the structure can be appropriately adapted to the shape of the teeth. The nano-fibrous structure may have a structure so that fixation to the teeth is obtained through adhesive properties of the structure, e.g. due to the surface area of the fibres of the nano-fibrous structure that can be in contact with the teeth. Applying the nano-fibrous structure may comprise pressing the nano-fibrous structure around the teeth. Applying the soaked nano-fibrous structure may comprise applying the nano-fibrous structure to the incisor teeth and the adjacent canine teeth so as to cover the tips of the canine teeth. The latter has the advantage that a homogeneous bleaching is obtained, whereby a plurality of teeth can be bleached, such as for example both the incisor teeth and the canine teeth at the same time. In some embodiments according to the present invention, the nano-fibrous structure may be adapted for, upon applying the soaked nano-fibrous structure, releasing teeth whitening moiety in a controlled way, e.g. at a controlled flow rate, to the teeth. The latter may for example be obtained by using a nano-fibrous structure with a varying diameter, porosity or density of the fibres used. The methods according to embodiments of the present invention furthermore may comprise one or more steps expressing the functionality of one or more components of the teeth whitening system as described in the fourth aspect.

The nano-fibrous structure may comprise fibres made of polyamide made by electrospinning using a mixture of formic acid and acetic acid. The ratio of formic acid and acetic acid may be between 90/10 and 10/90 weight percent, preferably between 30/70 and 70/30 weight percent and more preferably between 40/60 and 60/40 weight percent. It may advantageously be a 50/50 weight percent ratio. It is an advantage of a ratio of around 50/50 weight percent that a steady state continuous production and a high flow rate is possible.

By way of illustration, the present invention not being limited thereto, an exemplary method according to an embodiment of the present invention is described below. A method for whitening teeth thereby is illustrated, the method making use of a teeth whitening system as described in the first aspect. The teeth whitening system thereby comprises a nano-fibrous structure, in the present example being deposited on a substrate, and a teeth whitening moiety, in the present example being a teeth whitening gel in a plastic or glass container. The amount of teeth whitening gel thereby is between 1 to 2 ml. In the present example, the two components are stored in a plastic blister which is sealed with a plastic or cardboard film.

According to the exemplary method, the blister may be opened and the naofibrous structure and the teeth whitening moiety may be removed from the blister. The plastic or glass container then may be opened and the teeth whitening moiety may be pored in the blister, which may operate as a tray. The nano-fibrous structure, in the present example being deposited on a substrate, is then positioned in the blister. The latter advantageously is performed by providing the nano-fibrous structure towards the teeth whitening moiety, while maintaining the substrate, if present directing away from the teeth whitening moiety. The teeth whitening moiety may thus be soaked by the nano-fibrous structure. This process is completed in 2-5 minutes. During soaking of the teeth whitening moiety, preferably an initial contact between the teeth bleaching agent and the pH regulating agent is obtained as the pH regulating agent may be initially present in the nano-fibrous structure or may be separately stored from the teeth bleaching moiety and provided to the nano-fibrous structure before initiating the soaking. During the soaking, the teeth bleaching agent thus also may mix with the pH regulating compound which may result in increase of the pH to about 7-9. The soaked nano-fibrous structure then may be taken out of the blister and the system then may be positioned onto the front teeth by a slight pressure. This positioning may include covering the incisor teeth as well as the canine teeth, including their tips. Positioning may comprise folding parts, e.g. triangular parts, over the teeth and fixing them onto the backside of the teeth. This fixing may be performed by providing the slight pressure, whereby the adhesive effect is obtained by the structure of the nano-fibrous structure, without the need for adding an adhesive. The nano-fibrous structure deposited on a substrate is maintained at the teeth for a predetermined time. After treatment, the nano-fibrous structure deposited on a substrate may be removed by hand and the teeth may be rinsed with water or brushed.

In further aspects, the present invention also relates to the use of a teeth whitening system or a nano-fibrous structure according to any of the embodiments of the fourth and further aspect described above in a teeth whitening application. The advantages and features of the systems and/or structures described thereby may result in advantageous use of such systems and/or structures.

By way of illustration, embodiments of the present invention not being limited thereto, a number of examples of the particular embodiments for teeth bleaching devices is shown below.

### Example 10

The device comprises (1) a nano-fibrous structure deposited on a substrate, having dimensions of 7 by 2 cm, (2) a teeth whitening gel (1 mL) or solution filled in a plastic or glass container and a plastic blister containing (1) and (2) and sealed with a plastic or cardboard film. The blister is opened and components 1 and 2 are taken out. The plastic or glass container is opened and the gel is poured into the blister. The nano-fibrous structure deposited on a substrate is positioned in the blister with the nano-fibrous structure towards the gel (downward). The gel is soaked by the nano-fibrous structure. This process is completed in 2-5 minutes. During soaking of the gel or solution, it also mixes with the pH regulating compound, present in the nano-fibrous structure, which results in increase of the pH to about 8. The nano-fibrous structure deposited on a substrate is taken out of the blister and the full part is positioned onto the front teeth by a slight pressure. The triangular parts are then folded over the teeth and fixed onto the backside of the teeth as these are inherently adhesive due to the nano-fibrous structure. The nano-fibrous structure deposited on a substrate and containing the gel is maintained at the teeth for a period of 10 minutes. After treatment the nano-fibrous structure deposited on a substrate is removed by hand and the teeth are rinsed with water and/or brushed. This process is repeated twice a day for example for seven days.

The device as described above was used by 4 test persons according to the procedure described above. All test persons started with a teeth colour corresponding to A₃ on the VITA® shade scale. After 7 days of treatment the VITA shade of all teeth improved to A₁ shade, which is visually much more white than A₃. After 6 months the A₁ value is still maintained.

### Example 11

A teeth whitening gel that may be used in embodiments according to the present invention, and e.g. in the above described example 10 may be a gel for teeth whitening containing sorbitol 26; glycerol 25, hydrogen peroxide (35% solution) 20; Carboxymethylcellulose 0.10; Gantrez® 2.50; citric acid 0.04; sodium citrate 0.5, spear/peppermint 0.10 and water to obtain 100 m/m %. This composition results in a hydrogen peroxide concentration of 6%. The gel thus may be used with a device and according to a procedure as described in examples and/or embodiments of the present invention.

### Example 12

A teeth whitening gel that may be used in embodiments according to the present invention, and e.g. in the above described example 10 may be a gel for teeth whitening containing sorbitol 21; glycerol 20, hydrogen peroxide (35% solution) 40; Carboxymethylcellulose 0.10; Gantrez® 3.50; citric acid 0.04; sodium citrate 0.5, spear/peppermint 0.10 and water to obtain 100 m/m %. This composition results in a hydrogen peroxide concentration of 12%. The gel thus may be used with a device and according to a procedure as described in examples and/or embodiments of the present invention.

### Example 13

A teeth whitening gel that may be used in embodiments according to the present invention, and e.g. in the above described example 10 may be a gel for teeth whitening containing sorbitol 21; glycerol 20, hydrogen peroxide (35% solution) 50; Carboxymethylcellulose 0.10; Gantrez® 3.50; citric acid 0.04; sodium citrate 0.5, spear/peppermint 0.10 and water to obtain 100 m/m %. This composition results in a hydrogen peroxide concentration of 15%. The gel thus may be used with a device and according to a procedure as described in examples and/or embodiments of the present invention.

### Example 14

A teeth whitening gel that may be used in embodiments according to the present invention, and e.g. in the above described example 10 may be a gel for teeth whitening containing sorbitol 14.5; glycerol 13.9, hydrogen peroxide (35% solution) 66.7; Carboxymethylcellulose 0.10; Gantrez® 2.50; citric acid 0.04; sodium citrate 0.5, spear/peppermint 0.10 and water to obtain 100 m/m %. This composition results in a hydrogen peroxide concentration of 20%. The gel thus may be used with a device and according to a procedure as described in examples and/or embodiments of the present invention.

### Example 15

A teeth whitening gel that may be used in embodiments according to the present invention, and e.g. in the above described example 10 may be a gel for teeth whitening containing sorbitol 6; glycerol 6, hydrogen peroxide (35% solution) 83.3; Carboxymethylcellulose 0.10; Gantrez® 2.50; citric acid 0.04; sodium citrate 0.5, spear/peppermint 0.10 and water to obtain 100 m/m %. This composition results in a hydrogen peroxide concentration of 25%. The gel thus may be used with a device and according to a procedure as described in examples and/or embodiments of the present invention.

### Example 16

The examples as described above may for example be used in combination with a nano-fibrous structure comprising fibres made of polyamide, wherein the electrospinning is performed using a mixture of formic and acetic acid 50/50% as a solvent.

It is to be understood that although preferred embodiments, specific constructions and configurations, as well as materials, have been discussed herein for devices according to the present invention, various changes or modifications in form and detail may be made. For example, any formulas given above are merely representative of procedures that may be used. Functionality may be added or deleted from the block diagrams and operations may be interchanged among functional blocks. Steps may be added or deleted to methods described within the scope of the present invention. Whereas the present invention has been described with respect to a method for manufacturing, an electrospinning device and the resulting fibrous structures, the present invention also relates to a controller for controlling a relative distance between the outlets and the second planar surface for generating different properties between different layers in a fibrous structure.

1-26. (canceled)

27. An electrospinning device for producing fibrous structures, comprising:
a set of three or more neighbouring outlets arranged to output solution or melt, said three or more neighbouring outlets being arranged in a first plane;
a planar surface arranged parallel to said first plane, the planar surface arranged to receive output from said three or more neighbouring outlets, wherein said set of three or more neighbouring outlets and said planar surface are adapted to move relative to each other;
a voltage source arranged to provide a potential difference between said set of three or more neighbouring outlets and said planar surface;
a supply arrangement providing said solution or melt to said outlets;
two neighbouring outlets of said set of three or more neighbouring outlets being separated from each other by a distance of at least 4 cm.

28. The device according to claim 27, wherein the set of three or more neighbouring outlets are arranged in different rows, with outlets of adjacent rows being in a staggered configuration.

29. The device according to claim 27, wherein the set of three or more neighbouring outlets are arranged in different rows that are parallel but shifted with respect to their corresponding position of the outlets in the rows.

30. The device according to claim 27, wherein the distance between said three or more outlets is adapted to yield a fibrous structure comprising at least 50% of fibers substantially free of cross-links to neighboring fibers.

31. The device according to claim 27, wherein the distance between said three or more neighbouring outlets is adapted to yield a fibrous structure comprising at least 50% of fibres having segments that are substantially straight over a distance of 5 μm.

32. The device according to claim 27, wherein said two or more outlets are moveable relative to said surface both in a first direction and in a second direction that is different from said first direction.

33. A method for producing fibrous structures, comprising the steps:
(i) moving a set of three or more neighboring outlets outputting solution or melt, said set being disposed in a first plane, relative to a planar surface receiving output of said three or more neighbouring outlets;
(ii) applying a potential difference between said set of three or more neighbouring outlets and said planar surface; and
(iii) during said moving and applying, providing a solution or melt to said outlets;
wherein each two neighbouring outlets of said set of three or more neighbouring outlets are separated from one another by a distance of at least 4 cm.

34. An electrospun fibrous structure produced by using the method according to claim 33.

35. An electrospun fibrous structure, comprising at least 50% of fibres having segments that are substantially straight over a distance of 5 μm.

36. An electrospun fibrous structure according to claim 35, comprising at least 50% of fibers that are substantially cross-link free with respect to neighboring fibers.

37. An electrospun fibrous structure according to claim 35, comprising at least 50% of randomly oriented fibers.

38. An electrospun fibrous structure according to claim 35 having a porosity of at least 65%.

39. An electrospun fibrous structure according to claim 35 wherein 50% or more of the fibers have an average diameter between 3 and 2000 nm.

40. Use of an electrospun fibrous structure according to claim 35 for filtration or absorption.

41. Use of an electrospun fibrous structure according to claim 35 for teeth bleaching.
